(19) United States
(12) Patent Application Publication
(10) Pub. No.: US 2025/0109212 A1
(43) Pub. Date: Apr. 3, 2025

(54) RECOMBINANT FUSION PROTEIN TARGETING CD40 AND CD47

(71) Applicant: BIOSION INC., Nanjing, Jiangsu (CN)

(72) Inventors: Mingjiu CHEN, Nanjing (CN); Mark Zhiqing MA, Nanjing (CN); Yujie WANG, Lianyungang (CN); Jingjing XU, Lianyungang (CN)

(21) Appl. No.: 18/859,387

(22) PCT Filed: May 5, 2023
(86) PCT No.: PCT/CN2023/092215

§ 371 Date: Oct. 23, 2024

(30) Foreign Application Priority Data
May 6, 2022 (WO) PCT/CN2022/091058

(51) Int. Cl. C07K 16/28; A61K 38/00; C07K 14/705

(52) U.S. Cl. CPC C07K 16/2878; C07K 14/70503; A61K 38/00; C07K 2317/565; C07K 2317/76; C07K 2317/92; C07K 2317/94; C07K 2319/30; C07K 2319/74

(57) ABSTRACT

Provided is a recombinant fusion protein comprising (a) an anti-CD40 antibody or an antigen binding portion thereof comprising a heavy chain variable region and a light chain variable region, and (b) a CD47 binding domain. Provided is a nucleic acid molecule encoding the recombinant fusion protein, a vector comprising said nucleic acid and a host cell transformed or transfected with said vector or with such a nucleic acid. Furthermore, provided is a process for the production of said recombinant fusion protein, a medical use of said recombinant fusion protein and a kit comprising said recombinant fusion protein.

### INCORPORATION BY REFERENCE

This application claims priority to PCT application No. PCT/CN2022/091058 filed on May 6, 2022.

All documents cited or referenced herein (including without limitation all literature documents, patents, published patent applications cited herein) (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference. Any Genbank sequences mentioned in this disclosure are incorporated by reference with the Genbank sequence to be that of the earliest effective filing date of this disclosure.

### FIELD OF THE INVENTION

The present disclosure relates generally to a recombinant fusion protein, which binds to CD40 and CD47, with high affinity and functionality. A nucleic acid molecule encoding the recombinant fusion protein, an expression vector, a host cell and a method for expressing the recombinant fusion protein are also provided. The present disclosure further provides a pharmaceutical composition which may comprise the recombinant fusion protein, as well as a treatment method using the recombinant fusion protein of the disclosure.

### BACKGROUND OF THE INVENTION

### CD40 and CD40L

CD8⁺ cytotoxic T lymphocyte (CTL) responses play an important role in immunity against cancers and infections, while effective CTL responses are triggered by antigen specific T cell receptor binding with peptide-loaded major histocompatibility complex (MHC) on antigen presenting cells, followed by engagement of costimulatory molecules (Zhu Y et al., (2011) Immunity. 34 (4): 466-478).

CD40 and its major ligand CD40L are a pair of such costimulatory molecules. CD40 is a type I transmembrane protein that was firstly identified as a cell surface marker on B-lymphocytes and bladder tumor cells, and later found to be expressed on antigen presenting cells such as macrophages, dendritic cells and monocytes, while CD40L is primarily expressed by activated CD4⁺ T cells (Paulie S et al., (1989) J Immunol 142:590-595; Bereznaya N M et al., (2007) Exp Oncol. 29 (1): 2-12). When CD40 binds to CD40L, it recruits TRAF1 to TRAF6 to its cytoplasmic domains to drive signaling (Ma D Y et al., (2009) Semin Immunol 21 (5): 265-272). The CD40 signaling in dendritic cells may lead to upregulation of MHC molecules and costimulatory molecules as well as increased levels of T cell stimulatory cytokines such as interleukin 12, thus licensing dendritic cells and substituting “CD4⁺ T cell help” needed to drive CD8⁺ T cell responses (Ara A et al., (2018) Immunotargets Ther. 7:55-61). The immune activation by CD40 signaling may be independent of innate immune receptors such as Toll-like receptors (Byrne K T & Vonderheide R H (2016) Cell Rep. 15:2719-2732), and may convert cold tumors to hot ones, sensitizing them to checkpoint inhibition therapies (Vonderheide R H (2020) Annu Rev Med. 71:47-58).

CD40 expression was also found on tumor cells, including the B cell malignancies, melanoma, lung cancer, bladder cancer, gastric cancer, breast cancer and ovarian cancer, and studies showed that the CD40-CD40L interaction may inhibit tumor cell growth in e.g., melanoma and breast cancer (Von Leoprechting A et al., (1999) Cancer Res 59:1287-1294; Hirano A et al., (1999) Blood 93:2999-3007). In some cases, CD40 signaling in tumor cells was reported to promote tumor growth, but may at least enhance tumor antigen presentation.

Agonistic anti-CD40 antibodies have been developed for disease treatment. Selicrelumab (Pfizer and VLST), in its first-in-human single-dose study, has shown clinical efficacy in patients with advanced melanoma (Vonderheide R H et al., (2007) J. Clin. Oncol. 25:876-883; Bajor D L et al., (2014) Cancer Immunol. Res. 2:1051-1058).

Biologics that agonize CD40 signaling also showed efficacy in treating infectious diseases, including HIV-1/AIDS, tuberculosis and malaria (Elizabeth A Thompson, et al., (2015) J Immunol. 195 (3): 1015-1024).

### CD47 and SIRPα

CD47 is a transmembrane protein expressed on many types of cells, playing roles in e.g., cell proliferation, migration, and apoptosis (Ratnikova N M et al., (2017) Mol Biol. 51 (2): 251-261). Its ligands include the signal-regulatory protein alpha (SIRPα), also known as CD172a or Src homology 2 domain-containing phosphatase substrate-1, found on myeloid cells such as macrophages and dendritic cells. Upon engagement with SIRPα, CD47 acts as a “don't eat me” signal that prevents cell phagocytosis by macrophages, and cancer cells take advantage of such immune-tolerance mechanism to evade immune surveillance.

CD47 over-expression has been found in different types of tumors, including myeloma, leiomyosarcoma, acute lymphocytic leukemia (ALL), acute myeloid leukemia (AML), chronic myeloid leukemia (CML), non-Hodgkin's lymphoma, breast cancer, osteosarcoma, head and neck squamous cell carcinoma, small cell lung cancer, non-small cell lung cancer, multiple myeloma, melanoma, hepatocellular carcinoma, liver cancer, cholangiocarcinoma, ovarian cancer, bladder cancer, pancreatic ductal adenocarcinoma, and gastric carcinoma (including Epstein-Barr virus-associated gastric carcinoma), and associated with cancer metastasis and poor prognosis (Zhang W et al., (2020) Front Immunol. 11:18).

Studies have shown inhibiting the CD47-SIRPα signaling may inhibit tumor growth by inducing secretion of chemokines and cytokines that recruit more immune cells to tumors, triggering more macrophage-mediated tumor cell phagocytosis, promoting antigen-specific CD8⁺ T cell proliferation, reducing regulatory T cell number, increasing antibody-dependent cellular cytotoxicity such as NK cell-mediated cytotoxicity, and initiating tumor cell apoptosis via a caspase-independent mechanism (Weiskopf K et al., (2016) J Clin Investig. 126:2610-2620; Tseng D et al., (2013) Proc Natl Acad Sci USA. 110:11103-11108; Kim M J et al., (2008) Tumour Biol. 29:28-34). More particularly, the disruption of SIRPα-CD47 interaction may promote phagocytic uptake of cancer cells by macrophages, and the antigenic peptides from the cancer cells as generated during phagocytosis may later initiate an adaptive immune response (Chen, J. et al. (2017) 544:493-497).

Several CD47 targeting therapeutics, including the anti-CD47 antibodies and the SIRPα-Fc fusion proteins, are currently under clinical trials for treatment of both solid and hematopoietic tumors (Zhang W et al., (2020) supra). The SIRPα-Fc fusion proteins may contain a full-length SIRPα or one or more of its extracellular immunoglobulin superfamily domains.

CD47 upregulation and CD47's immune-suppressive role were also found on immune cells during viral and bacterial infections, and CD47 blockade by anti-CD47 antibodies promoted activation and effector functions of macrophages, dendritic cells and T cells (Cham L B et al., (2020) Antibodies (Basel) 9 (3): 44).

### Bi-Specific or Multi-Specific Fusion Proteins

As CD47 is ubiquitously expressed on human cells and with a high level on hematopoietic cells, monospecific anti-CD47 antibodies or proteins may bind normal cells, especially red blood cells that occupy about half of the blood volume, causing adverse side effects, including serious anemia, for which certain clinical trials were discontinued.

Bispecific or multi-specific fusion proteins may be designed to bind CD47 and another antigen (e.g., CD40) to more accurately target tumor and/or immune cells in microenvironments, resulting in reduced or eliminated off-target effects.

Citation or identification of any document in this application is not an admission that such document is available as prior art to the present invention.

### SUMMARY OF THE INVENTION

The inventors of the present application have designed and prepared recombinant fusion proteins that bind both CD40 and CD47, wherein the recombinant fusion proteins show a low aggregation level, and have comparable or higher binding affinity to human and monkey CD40 proteins, comparable or higher blocking activity on CD40-CD40L binding, comparable or higher blocking activity on CD47-SIRPα binding, comparable or higher agonistic activity on CD40 signaling, and comparable or higher capability to induce phagocytosis of CD47⁺ cells, as compared to their monospecific counterparts and the prior art antibodies such as Selicrelumab.

The recombinant fusion proteins of the disclosure may be used for in vitro and in vivo assays and treatment of diseases associated with CD40 and/or CD47 signaling such as tumors.

In a first aspect, the disclosure provides a recombinant fusion protein that binds both CD40 and CD47, which may comprise an anti-CD40 antibody or an antigen binding portion thereof, and a CD47 binding domain.

The anti-CD40 antibody or the antigen-binding portion thereof may comprise a heavy chain variable region and a light chain variable region. In certain embodiments, the anti-CD40 antibody or the antigen-binding portion thereof may comprise two identical heavy chain variable regions and two identical light chain variable regions. In certain embodiments, a heavy chain constant region is linked to the C-terminus of the heavy chain variable region, and optionally a light chain constant region is linked to the C-terminus of the light chain variable region.

The anti-CD40 antibody or the antigen-binding portion thereof may be a full-length antibody, a Fab fragment, a F(ab′)₂ fragment, a Fd fragment, or a Fv fragment.

The anti-CD40 antibody or the antigen-binding portion thereof may have agonistic activity on CD40 signaling.

The heavy chain variable region of the anti-CD40 antibody or the antigen-binding portion thereof may comprise a VH CDR1, a VH CDR2 and a VH CDR3 that may comprise the amino acid sequences of SEQ ID NOs: 1, 2 and 3, respectively. The heavy chain variable region may comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 7. The heavy chain variable region may comprise the amino acid sequence of SEQ ID NO: 7. The heavy chain constant region may be an IgG1, IgG2, or IgG4 heavy chain constant region, or a functional fragment thereof, such as an Fc fragment. The heavy chain constant region may be naturally occurring or engineered to have certain desired characteristics. In certain embodiments, the heavy chain constant region is human IgG1, IgG2 or IgG4 heavy chain constant region or a functional fragment thereof, having e.g., the amino acid sequence of SEQ ID NO: 9.

The light chain variable region of the anti-CD40 antibody or the antigen-binding portion thereof may comprise a VL CDR1, a VL CDR2 and a VL CDR3 that may comprise the amino acid sequences of SEQ ID NOs: 4, 5 and 6, respectively. The light chain variable region may comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 8. The light chain variable region may comprise the amino acid sequence of SEQ ID NO: 8. The light chain constant region may be a kappa or lambda light chain constant region. In certain embodiments, the light chain constant region may having e.g., the amino acid sequence of SEQ ID NO: 10.

The anti-CD40 antibody or the antigen-binding portion thereof may comprise the heavy chain variable region and the light chain variable region, wherein the heavy chain variable region comprises the VH CDR1, VH CDR2 and VH CDR3 and the light chain variable region comprises the VL CDR1, VL CDR2 and VL CDR3, wherein the VH CDR1, VH CDR2, VH CDR3, VL CDR1, VL CDR2 and VL CDR3 may comprise the amino acid sequences of SEQ ID NOs: 1, 2, 3, 4, 5 and 6, respectively. The heavy chain variable region may comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 7, and the light chain variable region may comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 8. In certain embodiments, the heavy chain variable region may comprise the amino acid sequence of SEQ ID NO: 7, and the light chain variable region may comprise the amino acid sequence of SEQ ID NO: 8.

In certain embodiments, the anti-CD40 antibody or the antigen-binding portion thereof may comprise a heavy chain and a light chain. In certain embodiments, the anti-CD40 antibody or the antigen-binding portion thereof may comprise two identical heavy chains and two identical light chains.

The heavy chain of the anti-CD40 antibody or the antigen-binding portion thereof may comprise a heavy chain variable region and a heavy chain constant region, wherein the heavy chain variable region and the heavy chain constant region may comprise amino acid sequences described above. The heavy chain may comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 19. The heavy chain may comprise the amino acid sequence of SEQ ID NO: 19.

The light chain of the anti-CD40 antibody or the antigen-binding portion thereof may comprise a light chain variable region and optionally a light chain constant region, wherein the light chain variable region and the light chain constant region may comprise amino acid sequences described above. The light chain may comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 20. The light chain may comprise the amino acid sequence of SEQ ID NO: 20.

The heavy chain may comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 19, and the light chain may comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 20. In certain embodiments, the heavy chain may comprise the amino acid sequence of SEQ ID NO: 19, and the light chain may comprise the amino acid sequence of SEQ ID NO: 20.

The CD47 binding domain may be a human SIRPα or the portion thereof. In some embodiments, the CD47 binding domain may be a first Ig-like extracellular domain of human SIRPα. In some embodiments, the CD47 binding domain is a SIRPα isoform 2 (SIRPαV2), of wild type, or with mutations. In certain embodiments, the CD47 binding domain is a first Ig-like extracellular domain of SIRPαV2 (SIRPαV2D1). In certain embodiments, the SIRPαV2D1 is a wild-type one having e.g., the amino acid sequence of SEQ ID NO: 11 (X1=V, X2=K, X3=S, X4=K, X5=F). In certain embodiments, the SIRPαV2D1 is a variant with higher CD47 binding affinity/capability, having e.g., the amino acid sequence of SEQ ID NO: 11 (X1=I, X2=R, X3=T, X4=K, X5=F; or X1=I, X2=R, X3=T, X4=R, X5=V).

The CD47 binding domain may be linked to the N- or C-terminus of the anti-CD40 antibody or antigen-binding portion thereof. In certain embodiments, the anti-CD40 antibody or antigen-binding portion thereof comprises a heavy chain variable region, a light chain variable region, a heavy chain constant region is linked to the C-terminus of the heavy chain variable region, and a light chain constant region is linked to the C-terminus of the light chain variable region, and the CD47 binding domain is linked to the N-terminus of the heavy chain variable region or light chain variable region, or to the C-terminus of the heavy chain constant region or light chain constant region. In certain embodiments, the CD47 binding domain is linked to the N-terminus of the heavy chain variable region, or to the C-terminus of the heavy chain constant region or light chain constant region. In certain embodiments, the anti-CD40 antibody or antigen-binding portion thereof comprises a heavy chain and a light chain, and the CD47 binding domain is linked to the N- or C-terminus of the heavy chain, or to the N- or C-terminus of the light chain. In certain embodiments, the anti-CD40 antibody or antigen-binding portion thereof comprises two identical heavy chains and two identical light chains, and the CD47 binding domain is linked to the N- or C-terminus of each heavy chain, or to the N- or C-terminus of each light chain. In certain embodiments, the anti-CD40 antibody or antigen-binding portion thereof comprises two identical heavy chains and two identical light chains, and the CD47 binding domain is linked to the N- or C-terminus of each heavy chain, or to the C-terminus of each light chain.

In certain embodiments, the CD47 binding domain may be linked to the anti-CD40 antibody or antigen-binding portion thereof via a linker. In certain embodiments, the CD47 binding domain is linked to the heavy chain variable region or the light chain variable region via a linker. In certain embodiments, the CD47 binding domain is linked to N-terminus of the heavy chain variable region or light chain variable region via a linker. In certain embodiments, the CD47 binding domain is linked to the heavy chain constant region or the light chain constant region via a linker. In certain embodiments, the CD47 binding domain is linked to C-terminus of the heavy chain constant region or light chain constant region via a linker. In certain embodiments, the CD47 binding domain is linked to the heavy chain or the light chain via a linker. In certain embodiments, the CD47 binding domain is linked to N- or C-terminus of heavy chain, or to the C-terminus of light chain via a linker. The linker may be a short peptide chain of 5 to 20 amino acid residues. In certain embodiments, the linker may be a GS linker, having e.g., the amino acid sequence of SEQ ID NOs: 12, 13, 14 or 15.

The recombinant fusion protein of the disclosure, in certain embodiments, may comprise:
i) an anti-CD40 heavy chain variable region-heavy chain constant region-linker-SIRPαV2D1 polypeptide chain comprising the amino acid sequence of SEQ ID NO: 16, and an anti-CD40 light chain variable region-light chain constant region polypeptide chain comprising the amino acid sequence of SEQ ID NO: 20;
ii) a SIRPαV2D1-linker-anti-CD40 heavy chain variable region-heavy chain constant region polypeptide chain comprising the amino acid sequence of SEQ ID NO: 17 (X1=V, X2=K, X3=S, X4=K, X5=F; X1=I, X2=R, X3=T, X4=K, X5=F; or X1=I, X2=R, X3=T, X4=R, X5=V), and an anti-CD40 light chain variable region-light chain constant region polypeptide chain comprising the amino acid sequence of SEQ ID NO: 20; or
iii) an anti-CD40 heavy chain variable region-heavy chain constant region polypeptide chain comprising the amino acid sequence of SEQ ID NO: 19, and an anti-CD40 light chain variable region-light chain constant region-linker-SIRPαV2D1 polypeptide chain comprising the amino acid sequence of SEQ ID NO: 18.

The recombinant fusion protein of the disclosure may comprise:
i) a first polypeptide chain and a second polypeptide chain each comprising the anti-CD40 heavy chain variable region, the heavy chain constant region, and the SIRPαV2D1, and a third polypeptide chain and a fourth polypeptide chain each comprising the anti-CD40 light chain variable region and optionally the light chain constant region; or
ii) a first polypeptide chain and a second polypeptide chain each comprising the anti-CD40 heavy chain variable region and the heavy chain constant region, and
a third polypeptide chain and a fourth polypeptide chain each comprising the anti-CD40 light chain variable region, optionally the light chain constant region, and the SIRPαV2D1,
wherein the heavy chain variable region in the first polypeptide chain and the light chain variable region in the third polypeptide chain associate to form an CD40 binding domain,
wherein the heavy chain variable region in the second polypeptide chain and the light chain variable region in the fourth polypeptide chain associate to form an CD40 binding domain,
wherein the heavy chain constant region in the first polypeptide chain and the heavy chain constant region in the second polypeptide chain are associated together.

In certain embodiments, the recombinant fusion protein may comprise:
i) a first polypeptide chain and a second polypeptide chain each, from the N terminus to the C terminus, comprising the anti-CD40 heavy chain variable region, the heavy chain constant region, and the SIRPαV2D1, and a third polypeptide chain and a fourth polypeptide chain each comprising the anti-CD40 light chain variable region;
ii) a first polypeptide chain and a second polypeptide chain each, from the N terminus to the C terminus, comprising the SIRPαV2D1, the anti-CD40 heavy chain variable region, and the heavy chain constant region, and
a third polypeptide chain and a fourth polypeptide chain each comprising the anti-CD40 light chain variable region; or
iii) a first polypeptide chain and a second polypeptide chain each, from the N terminus to the C terminus, comprising the anti-CD40 heavy chain variable region and the heavy chain constant region, and
a third polypeptide chain and a fourth polypeptide chain each, from the N terminus to the C terminus, comprising the anti-CD40 light chain variable region and the SIRPαV2D1,
wherein the heavy chain variable region in the first polypeptide chain and the light chain variable region in the third polypeptide chain associate to form an CD40 binding domain,
wherein the heavy chain variable region in the second polypeptide chain and the light chain variable region in the fourth polypeptide chain associate to form an CD40 binding domain,
wherein the heavy chain constant region in the first polypeptide chain and the heavy chain constant region in the second polypeptide chain are associated together.

The recombinant fusion protein of the disclosure, in certain embodiments, may comprise:
i) a first polypeptide chain and a second polypeptide chain each, from the N terminus to the C terminus, comprising the anti-CD40 heavy chain variable region, the heavy chain constant region, and the SIRPαV2D1, and
a third polypeptide chain and a fourth polypeptide chain each, from the N terminus to the C terminus, comprising the anti-CD40 light chain variable region and the light chain constant region;
ii) a first polypeptide chain and a second polypeptide chain each, from the N terminus to the C terminus, comprising the SIRPαV2D1, the anti-CD40 heavy chain variable region, and the heavy chain constant region, and
a third polypeptide chain and a fourth polypeptide chain each, from the N terminus to the C terminus, comprising the anti-CD40 light chain variable region and the light chain constant region; or
iii) a first polypeptide chain and a second polypeptide chain each, from the N terminus to the C terminus, comprising the anti-CD40 heavy chain variable region and the heavy chain constant region, and
a third polypeptide chain and a fourth polypeptide chain each, from the N terminus to the C terminus, comprising the anti-CD40 light chain variable region, the light chain constant region and the SIRPαV2D1,
wherein the heavy chain variable region in the first polypeptide chain and the light chain variable region in the third polypeptide chain associate to form an CD40 binding domain,
wherein the heavy chain variable region in the second polypeptide chain and the light chain variable region in the fourth polypeptide chain associate to form an CD40 binding domain,
wherein the heavy chain constant region in the first polypeptide chain and the heavy chain constant region in the second polypeptide chain are associated together.

The recombinant fusion protein of the disclosure, in certain embodiments, may comprise:
i) a first polypeptide chain and a second polypeptide chain each, from the N terminus to the C terminus, comprising the anti-CD40 heavy chain variable region, the heavy chain constant region, the linker and the SIRPαV2D1, and
a third polypeptide chain and a fourth polypeptide chain each, from the N terminus to the C terminus, comprising the anti-CD40 light chain variable region and the light chain constant region;
ii) a first polypeptide chain and a second polypeptide chain each, from the N terminus to the C terminus, comprising the SIRPαV2D1, the linker, the anti-CD40 heavy chain variable region, and the heavy chain constant region, and
a third polypeptide chain and a fourth polypeptide chain each, from the N terminus to the C terminus, comprising the anti-CD40 light chain variable region and the light chain constant region; or
iii) a first polypeptide chain and a second polypeptide chain each, from the N terminus to the C terminus, comprising the anti-CD40 heavy chain variable region and the heavy chain constant region, and
a third polypeptide chain and a fourth polypeptide chain each, from the N terminus to the C terminus, comprising the anti-CD40 light chain variable region, the light chain constant region, the linker and the SIRPαV2D1,
wherein the heavy chain variable region in the first polypeptide chain and the light chain variable region in the third polypeptide chain associate to form an CD40 binding domain,
wherein the heavy chain variable region in the second polypeptide chain and the light chain variable region in the fourth polypeptide chain associate to form an CD40 binding domain,
wherein the heavy chain constant region in the first polypeptide chain and the heavy chain constant region in the second polypeptide chain are associated together.

In certain embodiments, the recombinant fusion protein of the disclosure comprises:
i) a first polypeptide chain and a second polypeptide chain each comprising the amino acid sequence of SEQ ID NO: 16, and
a third polypeptide chain and a fourth polypeptide chain each comprising the amino acid sequence of SEQ ID NO: 20;
ii) a first polypeptide chain and a second polypeptide chain each comprising the amino acid sequence of SEQ ID NO: 17 (X1=V, X2=K, X3=S, X4=K, X5=F; X1=I, X2=R, X3=T, X4=K, X5=F; or X1=I, X2=R, X3=T, X4=R, X5=V), and
a third polypeptide chain and a fourth polypeptide chain each comprising the amino acid sequence of SEQ ID NO: 20; or
iii) a first polypeptide chain and a second polypeptide chain each comprising the amino acid sequence of SEQ ID NO: 19, and
a third polypeptide chain and a fourth polypeptide chain each comprising the amino acid sequence of SEQ ID NO: 18.

The present application also provides a nucleic acid molecule encoding the recombinant fusion protein of the disclosure, as well as an expression vector that may comprise such a nucleic acid, and a host cell transformed or transfected with such an expression vector or with such a nucleic acid. A method for preparing the recombinant fusion protein of the disclosure using the host cell is also provided, that may comprise steps of (i) expressing the recombinant fusion protein in the host cell and (ii) isolating the recombinant fusion protein from the host cell or its cell culture.

The present application also provides a pharmaceutical composition that may comprise the recombinant fusion protein, the nucleic acid molecule, the expression vector, or the host cell of the disclosure, and a pharmaceutically acceptable carrier. The pharmaceutical composition may further comprise an additional agent, such as an anti-tumor agent.

In a second aspect, the present application provides a method for treating a disease associated with CD40 and/or CD47 signaling in a subject in need thereof, which may comprise administering to the subject a therapeutically effective amount of the pharmaceutical composition of the disclosure.

The disease may be a cancer. The cancer may be a solid cancer or a hematopoietic cancer, including, but not limited to, leiomyosarcoma, acute lymphocytic leukemia (ALL), acute myeloid leukemia (AML), chronic myeloid leukemia (CML), non-Hodgkin's lymphoma, Hodgkin's lymphoma, chronic lymphocytic leukemia, colon cancer, kidney cancer, prostate cancer, cervix cancer, nasopharynx cancer, breast cancer, osteosarcoma, head and neck squamous cell carcinoma, lung cancer (including small cell lung cancer and non-small cell lung cancer), multiple myeloma, melanoma, hepatocellular carcinoma, liver cancer, cholangiocarcinoma, ovarian cancer, bladder cancer, pancreas cancer (including pancreatic ductal adenocarcinoma), and gastric carcinoma. In certain embodiments, the pharmaceutical composition of the disclosure may be used in combination with chemotherapy and checkpoint blockade therapy, such as an anti-PD-L1 antibody, an anti-PD-1 antibody, or an anti-CTLA-4 antibody.

The disease may be an infectious disease. The infectious disease may be caused by a bacterial, viral or parasitic infection.

In certain embodiments, the subject is human.

In a third aspect, the present application provides a method for enhancing an immune response in a subject in need thereof, comprising administering to the subject a therapeutically effective amount of the pharmaceutical composition of the disclosure. In certain embodiments, the subject is human.

The present application also provides a method for reversing or reducing an immune-suppression in a subject in need thereof, comprising administering to the subject a therapeutically effective amount of the pharmaceutical composition of the disclosure.

In certain embodiments, the subject is human.

Also provided is the use of the pharmaceutical composition of the disclosure in treating a disease associated with CD40 and/or CD47 signaling, in enhancing an immune response, and/or in reversing or reducing an immune-suppression.

In a fourth aspect, the present application provides a kit that may comprise the recombinant fusion protein, the pharmaceutical composition, the nucleic acid molecule, the expression vector, or the host cell of the disclosure. The term “kit” means two or more components-one of which corresponding to the recombinant fusion protein, the pharmaceutical composition, the nucleic acid molecule, the expression vector or the host cell of the disclosure-packaged together in a container, recipient or otherwise. A kit can hence be described as a set of products and/or utensils that are sufficient to achieve a certain goal, which can be marketed as a single unit.

The kit may comprise one or more recipients (such as vials, ampoules, containers, syringes, bottles, bags) of any appropriate shape, size and material (preferably waterproof, e.g. plastic or glass) containing the recombinant fusion protein or the pharmaceutical composition of the disclosure. The kit may additionally contain directions for use (e.g., in the form of a leaflet or instruction manual).

Other features and advantages of the instant disclosure will be apparent from the following detailed description and examples, which should not be construed as limiting. The contents of all references, Genbank entries, patents and published patent applications cited throughout this application are expressly incorporated herein by reference.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53 (c) EPC and Rule 28 (b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

### BRIEF DESCRIPTION OF THE DRAWINGS

The following detailed description, given by way of example, but not intended to limit the invention solely to the specific embodiments as described, may best be understood in conjunction with the accompanying drawings.

FIG. 1 shows the schematic diagrams of recombinant fusion protein structures.

FIG. 2 shows the binding activity of the recombinant fusion proteins of the disclosure to 293T-CD40 cells expressing human CD40 on the cell surface in a cell based binding FACS assay.

FIG. 3 shows the binding activity of the recombinant fusion proteins of the disclosure to 293F-CD47 cells expressing human CD47 on the cell surface in a cell based binding FACS assay.

FIG. 4 shows the ability of the recombinant fusion proteins of the disclosure to block human CD40-human CD40L binding in a competitive ELISA assay.

FIG. 5 shows the ability of the recombinant fusion proteins of the disclosure to block human CD47-human CD172a binding in a competitive ELISA assay.

FIG. 6 shows the ability of the recombinant fusion proteins of the disclosure to block human SIRPα binding to cell-surface human CD47 in a cell-based FACS assay.

FIG. 7 shows the ability of the recombinant fusion proteins of the disclosure to block human SIRPα binding to cell-surface human CD47 in a cell-based FACS assay.

FIG. 8 shows the CD40 agonistic activity of the recombinant fusion proteins of the disclosure in a cell based reporter assay.

FIG. 9 shows the ability of the recombinant fusion proteins of the disclosure to induce phagocytosis of human Jurkat cancer cells by macrophages in a cell-based assay.

FIG. 10 shows the binding activity of the recombinant fusion proteins of the disclosure to human CD40 and CD47 in a dual-binding ELISA assay.

### DETAILED DESCRIPTION OF THE INVENTION

To ensure that the present disclosure may be more readily understood, certain terms are first defined. Additional definitions are set forth throughout the detailed description.

The term “CD40” refers to cluster of differentiation 40. The term “CD40” comprises variants, isoforms, homologs, orthologs and paralogs. For example, an antibody specific for a human CD40 protein may, in certain cases, cross-react with a CD40 protein from a species other than human, such as monkey. In other embodiments, an antibody specific for a human CD40 protein may be completely specific for the human CD40 protein and exhibit no cross-reactivity to other species or of other types, or may cross-react with CD40 from certain other species but not all other species.

The term “human CD40” refers to a CD40 protein having an amino acid sequence from a human, such as the amino acid sequence of human CD40 having NCBI reference no. NP_001241.1 (Sasaki K et al., (2021) J Exp Clin Cancer Res 40 (1): 212). The term “monkey CD40” or “cyno CD40” refers to a CD40 protein having an amino acid sequence from a monkey species.

The term “SIRPα” refers to wild-type signal-regulatory protein alpha or a recombinant or non-recombinant polypeptide having the amino acid sequence of wild-type signal-regulatory protein alpha or a native or naturally occurring allelic variant of signal-regulatory protein alpha or an artificial variant of signal-regulatory protein alpha. In one embodiment, SIRPα is a wild-type mammalian SIRPα, whereas in a preferred embodiment, SIRPα is a wild-type human SIRPα. The term “human SIRPα” refers to a SIRPα protein having an amino acid sequence from a human, such as the amino acid sequence having GenBank accession no.: AAH75849.1 (Strausberg R. L. et al., (2002) Proc. Natl. Acad. Sci. U.S.A. 99 (26): 16899-16903). In one embodiment, SIRPα includes a signal sequence, whereas in another embodiment, SIRPα refers to the mature form of the protein. Ten human SIRPα alleles have been found till now, and the human SIRPα isoform 2 (or V2) was reported to have reduced or minimal binding affinity to red blood cells.

The term “bispecific” as used herein refers to a binding molecule which is a fusion protein and comprises at least a first and a second binding domain, wherein the first binding domain is capable of binding to one antigen or target, and the second binding domain is capable of binding to another antigen or target. Accordingly, a bispecific fusion protein according to the application comprise at least binding specificities for two different antigens or targets and are at least bispecific. The “bispecific fusion protein” of the application also comprises multi-specific binding molecules such as e.g., tri-specific binding molecules, the latter ones including three binding domains. It is also envisaged that the bispecific fusion protein of the application has, in addition to its function to bind to the target molecules CD40 and CD47, a further function.

The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

The term “immune suppression” or “immunosuppression” refers to the reduction of the immune system's activation or efficacy, caused by e.g., age, persisting disease, malnutrition, cancers, chemotherapy or radiotherapy, or the like. The immune suppression can be reversed in certain circumstances by e.g., manipulating some pathways.

The term “antibody” as used herein refers to an immunoglobulin molecule that recognizes and specifically binds a target, such as CD40, through at least one antigen-binding site wherein the antigen-binding site is usually within the variable region of the immunoglobulin molecule. As used herein, the term encompasses intact polyclonal antibodies, intact monoclonal antibodies, single-chain Fv (scFv) antibodies, heavy chain antibodies (HCAbs), light chain antibodies (LCAbs), multi-specific antibodies, bi-specific antibodies, monospecific antibodies, monovalent antibodies, fusion proteins comprising an antigen-binding site of an antibody, and any other modified immunoglobulin molecules comprising an antigen-binding site (e.g., dual variable domain immunoglobulin molecules) as long as the antibodies exhibit the desired biological activity. Antibodies also include, but are not limited to, mouse antibodies, chimeric antibodies, humanized antibodies, and human antibodies. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes. Unless expressly indicated otherwise, the term “antibody” as used herein includes the “antigen-binding portion” of the intact antibodies. An conventional IgG is a glycoprotein which may comprise two identical heavy (H) chains and two identical light (L) chains inter-connected by disulfide bonds. Each heavy chain may be comprised of a heavy chain variable region (abbreviated herein as V_{H}) and a heavy chain constant region C_{H}. The heavy chain constant region may be comprised of three domains, C_{H1}, C_{H2} and C_{H3}. Each light chain may be comprised of a light chain variable region (abbreviated herein as V_{L}) and a light chain constant region C_{L}. The light chain constant region may be comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (Clq) of the classical complement system. The “functional fragment” of the heavy chain constant region refers to a part of the constant region that maintains the desired characteristics, such as the binding affinity to Fc receptors and/or the complement system proteins.

The term “antigen-binding portion” of an antibody (or simply “antibody portion”), as used herein, refers to one or more fragments of an antibody that retain the ability to specifically bind to an antigen (e.g., a CD40 protein). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment which may comprise two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR); and (viii) a nanobody, a heavy chain variable region containing a single variable domain and two constant domains. Furthermore, although the two domains of the Fv fragment, V_{L} and V_{H}, are coded by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al., (1988) Science 242:423-426; and Huston et al., (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

The “agonistic” anti-CD40 antibody or antigen binding portion thereof or “agonistic activity” on CD40 signaling means the anti-CD40 antibody or antigen binding portion thereof can bind CD40 and activate/induce CD40 signaling to e.g., promote immune cell activation and proliferation as well as cytokine and chemokine production. The agonistic anti-CD40 antibody or antigen binding portion thereof may promote a tumor-bearing subject's innate and adaptive immune response to tumors, via elevated antigen presenting ability of APCs, activation of tumor specific CD4⁺ and CD8⁺ T cells, secretion of cytokines and chemokines by lymphocytes and monocytes, enhanced tumor cell killing by cytotoxic lymphocytes and NK cells, etc.

As used herein, a recombinant fusion protein or a bispecific fusion protein that “specifically binds to CD40 (such as human CD40)” is intended to refer to a fusion protein that binds to the CD40 protein (human CD40 and possibly a CD40 protein from one or more non-human species) but does not substantially bind to non-CD40 proteins. Similarly, a fusion protein that “specifically binds to CD47 (such as human CD47)” refers to a fusion protein that binds to the CD47 protein from human or another non-human species but does not binds to non-CD47 proteins. Preferably, the fusion protein binds to CD40 or CD47 with “high affinity”, namely with a K_{D} of 5.0×10⁻⁸ M or less, and more preferably 1.0×10⁻⁸ M or less.

The term “does not substantially bind” to a protein or cells, as used herein, means does not bind or does not bind with a high affinity to the protein or cells, i.e. binds to the protein or cells with a K_{D} of 1.0×10⁻⁶ M or more, more preferably 1.0×10⁻⁵ M or more, more preferably 1.0×10⁻⁴ M or more, more preferably 1.0×10⁻³ M or more, even more preferably 1.0×10⁻² M or more.

The term “high affinity” refers to a K_{D} of 1.0×10⁻⁶ M or less, more preferably 1.0×10⁻⁸ M or less, even more preferably 6.0×10⁻⁹ M or less, and even more preferably 1.0×10⁻⁹ M or less for a target antigen.

The term “K_{assoc}” or “Kₐ”, as used herein, is intended to refer to the association rate of a particular protein-protein interaction, such as an antibody-antigen or a receptor-ligand interaction, whereas the term “K_{dis}” or “Kₐ”, as used herein, is intended to refer to the dissociation rate of a particular protein-protein interaction. The term “K_{D}”, as used herein, is intended to refer to the dissociation constant, which is obtained from the ratio of Kₐ to Kₐ (i.e., Kₐ/Kₐ) and is expressed as a molar concentration (M). K_{D} values can be determined using methods well established in the art. A preferred method for determining the K_{D} is by using surface plasmon resonance, preferably using a biosensor system such as a Biacore™ system.

The term “EC₅₀”, also known as half maximal effective concentration, refers to the concentration of a fusion protein of the disclosure which induces a response halfway between the baseline and maximum after a specified exposure time.

The term “IC₅₀”, also known as half maximal inhibitory concentration, refers to the concentration of a fusion protein of the disclosure which inhibits a specific biological or biochemical function by 50% relative to the absence of the fusion protein.

The term “subject” includes any human or nonhuman animal. The term “nonhuman animal” includes all vertebrates, e.g., mammals and non-mammals, such as non-human primates, sheep, dogs, cats, cows, horses, chickens, amphibians, and reptiles, although mammals are preferred, such as mice, rats, non-human primates, sheep, dogs, cats, cows and horses.

The term “therapeutically effective amount” means an amount of the fusion protein of the present disclosure sufficient to prevent or ameliorate the symptoms associated with a disease or condition (such as a cancer) and/or lessen the severity of the disease or condition. A therapeutically effective amount is understood to be in context to the condition being treated, where the actual effective amount is readily discerned by those of skill in the art.

The percent “identity” as used herein in the context of two or more nucleic acids or polypeptides, refers to two or more sequences or subsequences that are the same or have a specified percentage of nucleotides or amino acid residues that are the same, when compared and aligned (introducing gaps, if necessary) for maximum correspondence, considering or not considering conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides of the disclosure are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Various aspects of the disclosure are described below in further detail.

The recombinant fusion protein of the disclosure may comprise: (a) an anti-CD40 antibody or an antibody binding portion thereof, and (b) a CD47 binding domain. The CD47 binding domain may be linked, optionally via a linker, to the anti-CD40 antibody or antigen binding portion thereof.

The recombinant fusion protein of the disclosure show a low aggregation level, and has comparable or higher binding affinity to human and monkey CD40 proteins, comparable or higher blocking activity on CD40-CD40L binding, comparable or higher blocking activity on CD47-SIRPα binding, comparable or higher agonistic activity on CD40 signaling, and comparable or higher capability to induce phagocytosis of CD47⁺ cells (e.g. cancer cells including Jurkat cells or HL-60 cells), as compared to its monospecific counterparts and the prior art antibodies such as Selicrelumab.

The three components in the recombinant fusion protein of the present application are the CD47 binding domain, the linker, and the anti-CD40 antibody or antigen binding portion thereof. A person of ordinary skills in the art will recognize that there are many design choices for selecting the above three components. The CD47 binding domain may be linked to e.g., the N- or C-terminus of the anti-CD40 antibody or antigen binding portion thereof. Preferably, human-derived sequence is used in human cancer therapies, as the strong immunogenicity of the proteins or peptides from non-human animals may lead to allergy and other adverse effects. However, other animal proteins or peptides, humanized if appropriate, may also be used in the present application based on different application purposes.

The CD47 binding domain may be any protein or peptide capable of binding CD47, such as SIRPα, SIRPα variant, or an affinity optimized variant of SIRPα. A “variant” of SIRPα is defined as a SIRPα amino acid sequence that is altered by one or more amino acids as compared to wild-type SIRPα. The variant may have “conservative” changes, wherein a substituted amino acid has similar structural or chemical properties, e.g., replacement of leucine with isoleucine. More rarely, a variant can have “non-conservative” changes, e.g., replacement of a glycine with a tryptophan. Similar minor variations can also include amino acid deletions or insertions, or both. In one embodiment, SIRPα variants include polypeptides that have at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity with wild-type SIRPα.

In certain embodiments, the CD47 binding domain may be a first Ig-like extracellular domain of human SIRPα isoform 2 (SIRPαV2D1). The SIRPαV2D1 may be a wild-type SIRPαV2D1 or a SIRPαV2D1 variant having been engineered to possess higher CD47 binding affinity/capability. In certain embodiments, the SIRPαV2D1 may be a wild-type one comprising an amino acid sequence having at least 95% sequence identity to SEQ ID NO: 11 (X1=V, X2-K, X3=S, X4-K, X5=F). In certain embodiments, the SIRPαV2D1 may be a wild-type one comprising the amino acid sequence of SEQ ID NO: 11 (X1=V, X2=K, X3=S, X4=K, X5=F). In certain embodiments, the SIRPαV2D1 may be a SIRPαV2D1 variant comprising the amino acid sequence having at least 95% sequence identity to SEQ ID NO: 11 (X1=I, X2=R, X3=T, X4=K, X5=F; or X1=I, X2=R, X3=T, X4=R, X5=V). In certain embodiments, the SIRPαV2D1 may be a SIRPαV2D1 variant comprising the amino acid sequence of SEQ ID NO: 11 (X1=I, X2=R, X3=T, X4=K, X5=F; or X1=I, X2=R, X3=T, X4=R, X5=V). A “variant” of SIRPαV2D1 is defined as a SIRPαV2D1 amino acid sequence that is altered by one or more amino acids as compared to wild-type SIRPαV2D1. The variant may have “conservative” changes, wherein a substituted amino acid has similar structural or chemical properties, e.g., replacement of leucine with isoleucine. More rarely, a variant can have “non-conservative” changes, e.g., replacement of a glycine with a tryptophan. Similar minor variations can also include amino acid deletions or insertions, or both. In one embodiment, SIRPαV2D1 variants include polypeptides that have at least about 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity with wild-type SIRPαV2D1.

Linkers serve primarily as a spacer between the CD47 binding domain and the anti-CD40 antibody or antigen binding portion thereof. The linker may be made up of amino acids linked together by peptide bonds, preferably from 5 to 30 amino acids, from 10 to 30 amino acids, from 10 to 20 amino acids, or 15 amino acids, linked by peptide bonds, wherein the amino acids are selected from the 20 naturally occurring amino acids. One or more of these amino acids may be glycosylated, as is understood by those of skill in the art. In one embodiment, the 5 to 30 amino acids may be selected from glycine, alanine, proline, asparagine, glutamine, serine and lysine. In one embodiment, a linker is made up of a majority of amino acids that are sterically unhindered, such as glycine and alanine. Exemplary linkers are polyglycines (particularly (Glys, poly(Gly-Ala), and polyalanine, such as -GGGGS-(SEQ ID NO: 12), -GGGGSGGGGS-(SEQ ID NO: 13), -GGGGSGGGGSGGGGS-(SEQ ID NO: 14), and -GGGGSGGGGSGGGGSGGGGS-(SEQ ID NO: 15). Linkers may also be non-peptide linkers. For example, alkyl linkers such as —NH—, —(CH₂)s-C(O)—, wherein s=2-20 can be used. These alkyl linkers may further be substituted by any non-sterically hindering group such as lower alkyl (e.g., C₁₋₄) lower acyl, halogen (e.g., CI, Br), CN, NH₂, phenyl, etc. In other embodiment, the recombinant fusion protein of the present application can be assembled in the absence of linker.

The anti-CD40 antibody may be an isolated monoclonal antibody, such as the one disclosed in WO2021/197335. The anti-CD40 antibody or antigen binding portion thereof of the disclosure may be humanized.

The anti-CD40 antibody or the antigen-binding portion thereof of the disclosure may comprise a heavy chain variable region and a light chain variable region. In certain embodiments, the anti-CD40 antibody or the antigen-binding portion thereof may comprise two identical heavy chain variable regions and two identical light chain variable regions. In certain embodiments, a heavy chain constant region is linked to C-terminus of the heavy chain variable region, and optionally a light chain constant region is linked to C-terminus of the light chain variable region.

The anti-CD40 antibody or the antigen-binding portion thereof of the disclosure may comprise a heavy chain and a light chain. In certain embodiments, the anti-CD40 antibody or the antigen-binding portion thereof of the disclosure comprises two identical heavy chains and two identical light chains. The heavy chain may comprise a heavy chain variable region and a heavy chain constant region. The light chain may comprise a light chain variable region and an optional a light chain constant region.

The heavy chain variable region of the disclosure may comprise a VH CDR1, a VH CDR2 and a VH CDR3 that may comprise the amino acid sequences of SEQ ID NOs: 1, 2 and 3, respectively. The heavy chain variable region may comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 7.

The heavy chain constant region may be human IgG1, IgG2 or IgG4 heavy chain constant region, optionally engineered to have altered functional properties e.g., altered Fc receptor binding affinity. The heavy chain constant region may comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% sequence identity to SEQ ID NO: 9. In certain embodiments, the heavy chain constant region may comprise the amino acid sequence of SEQ ID NO: 9.

The light chain variable region of the disclosure may comprise a VL CDR1, a VL CDR2 and a VL CDR3 that may comprise the amino acid sequences of SEQ ID NOs: 4, 5 and 6, respectively. The light chain variable region may comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 8. The light chain constant region may be human kappa or lambda light chain constant region. The light chain constant region may comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 10. In certain embodiments, the light chain constant region may comprise the amino acid sequence of SEQ ID NO: 10.

The heavy chain variable region may comprise a VH CDR1, a VH CDR2 and a VH CDR3 that may comprise the amino acid sequences of SEQ ID NOs: 1, 2 and 3, respectively, and the light chain variable region may comprise a VL CDR1, a VL CDR2 and a VL CDR3 that may comprise the amino acid sequences of SEQ ID NOs: 4, 5 and 6, respectively. The heavy chain variable region may comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 7, and the light chain variable region may comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 8. In certain embodiments, the heavy chain variable region may comprise the amino acid sequence of SEQ ID NO: 7, and the light chain variable region may comprise the amino acid sequence of SEQ ID NO: 8. The heavy chain constant region may comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 9, and the light chain constant region may comprise an amino acid sequence having at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity to SEQ ID NO: 10. In certain embodiments, the heavy chain constant region may comprise the amino acid sequence of SEQ ID NO: 9, and the light chain constant region may comprise the amino acid sequence of SEQ ID NO: 10. In certain embodiments, the heavy chain variable region may comprise the amino acid sequence of SEQ ID NO: 7, the light chain variable region may comprise the amino acid sequence of SEQ ID NO: 8, the heavy chain constant region may comprise the amino acid sequence of SEQ ID NO: 9, and the light chain constant region may comprise the amino acid sequence of SEQ ID NO: 10.

The recombinant fusion protein of the disclosure may comprise:
i) a first polypeptide chain and a second polypeptide chain each comprising the anti-CD40 heavy chain variable region, the heavy chain constant region, and the SIRPαV2D1, and
a third polypeptide chain and a fourth polypeptide chain each comprising the anti-CD40 light chain variable region and optionally the light chain constant region; or
ii) a first polypeptide chain and a second polypeptide chain each comprising the anti-CD40 heavy chain variable region and the heavy chain constant region, and
a third polypeptide chain and a fourth polypeptide chain each comprising the anti-CD40 light chain variable region, optionally the light chain constant region, and the SIRPαV2D1,
wherein the heavy chain variable region in the first polypeptide chain and the light chain variable region in the third polypeptide chain associate to form an CD40 binding domain,
wherein the heavy chain variable region in the second polypeptide chain and the light chain variable region in the fourth polypeptide chain associate to form an CD40 binding domain,
wherein the heavy chain constant region in the first polypeptide chain and the heavy chain constant region in the second polypeptide chain are associated together.

The CD47 binding domain may be linked to the N- or C-terminus of the anti-CD40 antibody or the antigen-binding portion thereof. In certain embodiments, the anti-CD40 antibody or antigen-binding portion thereof comprises heavy chain variable region and light chain variable region, wherein a heavy chain constant region is linked to C-terminus of the heavy chain variable region, a light chain constant region is linked to C-terminus of the light chain variable region, and the CD47 binding domain is linked to the N-terminus of the heavy chain variable region or light chain variable region, or to the C-terminus of the heavy chain constant region or light chain constant region. In certain embodiments, the anti-CD40 antibody or antigen-binding portion thereof comprises heavy chain and light chain, and the CD47 binding domain is linked to the N or C terminus of the heavy chain, or to the N- or C-terminus of the light chain, of the anti-CD40 antibody or antigen binding portion thereof.

The recombinant fusion protein of the disclosure may be engineered by modifying one or more residues within one or both variable regions (i.e., V_{H} and/or V_{L}), for example within one or more CDR regions and/or within one or more framework regions of the anti-CD40 antibody or antigen binding portion thereof. Additionally or alternatively, the recombinant fusion protein can be engineered by modifying the residues within the constant region(s) of the anti-CD40 antibody or antigen binding portion thereof, for example to alter the effector function(s) of the recombinant fusion protein. Additionally, the recombinant fusion protein may be modified at residues at the CD47 binding domain, to alter the binding affinity to CD47 or other functional properties.

In certain embodiments, CDR grafting can be used to engineer variable regions of the anti-CD40 antibody or antigen binding portion thereof. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of the parent antibodies by constructing expression vectors that include CDR sequences from the parent antibody grafted onto framework sequences from a different antibody with different properties (see, e.g., Riechmann et al., (1998) Nature 332:323-327; Jones et al., (1986) Nature 321:522-525; Queen et al., (1989) Proc. Natl. Acad. See also U.S.A. 86:10029-10033; U.S. Pat. Nos. 5,225,539; 5,530,101; 5,585,089; 5,693,762 and 6,180,370).

Accordingly, another embodiment of the disclosure pertains to a recombinant fusion protein, which may comprise an anti-CD40 heavy chain variable region that may comprise VH CDR1, VH CDR2, and VH CDR3 sequences which may comprise the sequences of the present disclosure, as described above, and/or an anti-CD40 light chain variable region which may comprise the VL CDR1, VL CDR2, and VL CDR3 sequences which may comprise the sequences of the present disclosure, as described above. While these fusion protein contain the V_{H} and V_{L} CDR sequences of the anti-CD40 antibody of the present disclosure, they can contain different framework sequences.

Such framework sequences can be obtained from public DNA databases or published references that include germline antibody gene sequences. For example, germline DNA sequences for human heavy and light chain variable region genes can be found in the “VBase” human germline sequence database. As another example, the germline DNA sequences for human heavy and light chain variable region genes can be found in the Genbank database.

Antibody protein sequences are compared against a compiled protein sequence database using one of the sequence similarity searching methods called the Gapped BLAST (Altschul et al., (1997), supra), which is well known to those skilled in the art.

Preferred framework sequences for use in the anti-CD40 antibodies of the disclosure are those that are structurally similar to the framework sequences used by the antibodies of the disclosure. The VH CDR1, VH CDR2, and VH CDR3 sequences can be grafted onto framework regions that have the identical sequence as that found in the germline immunoglobulin gene from which the framework sequence derives, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370).

In addition, or as an alternative to modifications made within the framework or CDR regions, the anti-CD40 antibody or antigen binding portion thereof in recombinant fusion protein of the disclosure can be engineered to include modifications within the Fc region, typically to alter one or more functional properties of the recombinant fusion protein, such as serum half-life, complement fixation, Fc receptor binding, and/or antigen-dependent cellular cytotoxicity. Furthermore, a recombinant fusion protein of the disclosure can be chemically modified (e.g., one or more chemical moieties can be attached to the fusion protein) or be modified to alter its glycosylation, again to alter one or more functional properties.

In one embodiment, the hinge region of C_{H1} is modified such that the number of cysteine residues in the hinge region is altered, e.g., increased or decreased. This approach is described further in U.S. Pat. No. 5,677,425. The number of cysteine residues in the hinge region of C_{H1} is altered to, for example, facilitate assembly of the light and heavy chains or to increase or decrease the stability of the antibody.

In another embodiment, the Fc hinge region of the anti-CD40 antibody or the antigen binding portion thereof in recombinant fusion protein of the disclosure is mutated to alter the biological half-life of the recombinant fusion protein. Specifically, in another embodiment, one or more amino acid mutations are introduced into the C_{H2}-C_{H3} domain interface region of the Fc-hinge fragment such that the fusion protein has impaired Staphylococcyl protein A (SpA) binding relative to native Fc-hinge domain SpA binding. This approach is described in further detail in U.S. Pat. No. 6,165,745.

Alteration of amino acids near the junction of the Fc portion and the non-Fc portion can significantly increase the serum half-life of the Fc fusion protein. Thus, the junction region of the recombinant fusion protein of the disclosure may comprise changes relative to the sequences of naturally occurring immunoglobulin heavy chains, preferably within about 10 amino acids of the junction. These amino acid changes can result in increased hydrophobicity. In one embodiment, the constant region is derived from an IgG sequence in which the C-terminal lysine residue is replaced. Preferably, the C-terminal lysine of the IgG sequence is replaced with a non-lysine amino acid (e.g., alanine or leucine) to further increase serum half-life.

In still another embodiment, the glycosylation of the anti-CD40 antibody or antigen binding portion thereof in recombinant fusion protein is modified. Glycosylation can be altered to, for example, increase the affinity of the anti-CD40 antibody or antigen binding portion thereof for the antigen. Such carbohydrate modifications can be accomplished by, for example, altering one or more sites of glycosylation within the anti-CD40 antibody or antigen binding portion sequence. For example, one or more amino acid substitutions can be made that result in elimination of one or more variable region framework glycosylation sites to thereby eliminate glycosylation at that site. Such aglycosylation may increase the affinity of the antibody for antigen. See, e.g., U.S. Pat. Nos. 5,714,350 and 6,350,861.

Another modification of the anti-CD40 antibody or antigen binding portion thereof in recombinant fusion protein herein that is contemplated by this disclosure is pegylation. An antibody can be pegylated to, for example, increase the biological (e.g., serum) half-life of the antibody. As used herein, the term “polyethylene glycol” is intended to encompass any of the forms of PEG that have been used to derivatize other proteins, such as mono(C₁-C₁₀)alkoxy- or aryloxy-polyethylene glycol or polyethylene glycol-maleimide. Methods for pegylating proteins are known in the art and can be applied to the antibodies of the disclosure. See, e.g., EP 0 154 316 and EP 0 401 384.

The CD47 binding domain in the recombinant fusion protein of the disclosure may be modified to possess higher CD47 binding affinity. In some embodiments, a SIRPαV2D1 variant includes one or more mutations in the SIRPαV2D1 domain as compared to wild-type SIRPαV2D1. To determine the affinity of a SIRPαV2D1 variant for CD47 binding, surface plasmon resonance (SPR) may be used. In an alternative method to determine the avidity of a SIRPαV2D1 variant for CD47 binding, a cell binding assay may be used. When mutations are introduced into SIRPαV2D1 or the recombinant fusion protein of the disclosure, the resulting variant or fusion protein generally has enough SIRPα biological activity to be useful as a therapeutic protein. In some embodiments, the biological activity of the SIRPαV2D1 variant is at least 0.01 fold, 0.03 fold, 0.06 fold, 0.1 fold, 0.3 fold, 0.6 fold, 1 fold, 3 fold, 5, fold, 6 fold, 10 fold, 20 fold, 30 fold, 40 fold, 50 fold, 60 fold or 100 fold of the biological activity of wild type SIRPαV2D1 or a fusion protein containing wild-type SIRPαV2D1. Biological activity of the SIRPαV2D1 variants can be tested in an in vitro or in vivo assay. In vitro assays to determine the biological activity of SIRPαV2D1 on a cell expressing CD47 are well established in the art. For example, the biological activity may be determined in a leukocyte transmigration assay, as described by Liu et. al. (J. Mol. Bio., 365:680, 2007).

In another aspect, the disclosure provides a nucleic acid molecule that encodes the recombinant fusion protein of the disclosure. In certain embodiments, the disclosure provides nucleic acid molecules encoding i) an anti-CD40 heavy chain variable region-heavy chain constant region-linker-SIRPαV2D1 polypeptide chain comprising the amino acid sequence of SEQ ID NO: 16, and an anti-CD40 light chain variable region-light chain constant region polypeptide chain comprising the amino acid sequence of SEQ ID NO: 20; ii) a SIRPαV2D1-linker-anti-CD40 heavy chain variable region-heavy chain constant region polypeptide chain comprising the amino acid sequence of SEQ ID NO: 17 (X1=V, X2=K, X3=S, X4=K, X5=F; X1=I, X2=R, X3=T, X4=K, X5=F; or X1=I, X2=R, X3=T, X4=R, X5=V), and an anti-CD40 light chain variable region-light chain constant region polypeptide chain comprising the amino acid sequence of SEQ ID NO: 20; or iii) an anti-CD40 heavy chain variable region-heavy chain constant region polypeptide chain comprising the amino acid sequence of SEQ ID NO: 19, and an anti-CD40 light chain variable region-light chain constant region-linker-SIRPαV2D1 polypeptide chain comprising the amino acid sequence of SEQ ID NO: 18. The nucleic acids can be present in whole cells, in a cell lysate, or in a partially purified or substantially pure form. A nucleic acid is “isolated” or “rendered substantially pure” when purified from other cellular components or other contaminants, e.g., other cellular nucleic acids or proteins, by standard techniques. A nucleic acid of the disclosure can be, e.g., DNA or RNA and may or may not contain intronic sequences. In a preferred embodiment, the nucleic acid is a DNA molecule.

Nucleic acids of the disclosure can be obtained using standard molecular biology techniques. For example, the nucleic acid molecules of the disclosure may be chemically synthesized.

The present disclosure also provides an expression vector comprising the nucleic acid molecules of the disclosure. Examples of vectors include but are not limited to plasmids, viral vectors, yeast artificial chromosomes (YACs), bacterial artificial chromosomes (BACs), transformation-competent artificial chromosomes (TACs), mammalian artificial chromosomes (MACs) and human artificial episomal chromosomes (HAECs). The present disclosure further provides host cells transformed or transfected with the expression vectors or with the nucleic acid of the disclosure. Suitable host cells include Escherichia coli, yeasts and other eukaryotes. In one embodiment, DNAs encoding the polypeptide chains forming each recombinant fusion protein of the disclosure are inserted into one or more expression vectors such that the genes are operatively linked to transcriptional and translational regulatory sequences. In this context, the term “operatively linked” is intended to mean that the coding nucleotides are ligated into a vector such that transcriptional and translational control sequences within the vector serve their intended functions of regulating the transcription and translation of the recombinant fusion protein gene(s).

The term “regulatory sequence” is intended to include promoters, enhancers and other expression control elements (e.g., polyadenylation signals) that control the transcription or translation of the nucleotides. Such regulatory sequences are described, e.g., in Goeddel (Gene Expression Technology. Methods in Enzymology 185, Academic Press, San Diego, Calif. (1990)). Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, e.g., the adenovirus major late promoter (AdMLP) and polyomavirus enhancer. Alternatively, non-viral regulatory sequences can be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1 (Takebe et al., (1988) Mol. Cell. Biol. 8:466-472). The expression vector and expression control sequences are chosen to be compatible with the expression host cell used.

In addition to the recombinant fusion protein coding nucleotides and regulatory sequences, the expression vectors of the disclosure can carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216; 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr-host cells with methotrexate selection/amplification) and the neo gene (for G418 selection).

For expression of the peptide chains constituting the recombinant fusion protein, the expression vector(s) encoding the peptide chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the recombinant fusion proteins of the disclosure in either prokaryotic or eukaryotic host cells, expression in eukaryotic cells, and most preferably mammalian host cells, is the most preferred because such eukaryotic cells, and in particular mammalian cells, are more likely than prokaryotic cells to assemble and secrete a properly folded and immunologically active recombinant fusion protein.

Preferred mammalian host cells for expressing the recombinant fusion protein of the disclosure include Chinese Hamster Ovary (CHO cells) (including dhfr-CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) J. Mol. Biol. 159:601-621), NSO myeloma cells, COS cells and SP2 cells. In particular for use with NSO myeloma cells, another preferred expression system is the GS gene expression system disclosed in WO 87/04462, WO 89/01036 and EP 338,841. When recombinant expression vectors are introduced into mammalian host cells, the recombinant fusion proteins are produced by culturing the host cells for a period of time sufficient to allow for expression of the recombinant fusion protein in the host cells or, more preferably, secretion of the recombinant fusion protein into the culture medium in which the host cells are grown. The recombinant fusion proteins can be recovered from the culture medium using standard protein purification methods.

In another aspect, the present disclosure provides a pharmaceutical composition which may comprise the recombinant fusion protein, the nucleic acid molecule, the expression vector, and/or the host cell of the present disclosure formulated together with a pharmaceutically acceptable carrier. The recombinant fusion protein, the nucleic acid molecule, the expression vector, and/or the host cell can be dosed separately when the pharmaceutical composition contains more than one recombinant fusion protein, nucleic acid molecule, expression vector or host cell. The pharmaceutical composition may optionally contain one or more additional pharmaceutically active ingredients, such as another antibody or a drug, such as an anti-tumor drug.

The pharmaceutical composition may comprise any number of excipients. Excipients that can be used include carriers, thickening or emulsifying agents, solid binders, dispersion or suspension aids, solubilizers, colorants, flavoring agents, coatings, disintegrating agents, lubricants, sweeteners, preservatives, and combinations thereof. The selection and use of suitable excipients are taught in Gennaro, ed., Remington: The Science and Practice of Pharmacy, 20th Ed. (Lippincott Williams & Wilkins 2003), the disclosure of which is incorporated herein by reference.

Preferably, the pharmaceutical composition is suitable for intravenous, intramuscular, subcutaneous, parenteral, spinal or epidermal administration (e.g., by injection or infusion). Depending on the route of administration, the active ingredient can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intra-articular, subcapsular, subarachnoid, intraspinal, epidural and intrasternal injection and infusion. Alternatively, the pharmaceutical composition of the disclosure can be administered via a non-parenteral route, such as a topical, epidermal or mucosal route of administration, e.g., intranasally, orally, vaginally, rectally, sublingually or topically.

Pharmaceutical compositions can be in the form of sterile aqueous solutions or dispersions. They can also be formulated in a microemulsion, liposome, or other ordered structure suitable to high drug concentration.

A “therapeutically effective dosage” of the recombinant fusion protein, nucleic acid molecule, expression vector or host cell of the disclosure preferably results in a decrease in severity of disease symptoms, an increase in frequency and duration of disease symptom-free periods, or a prevention of impairment or disability due to the disease affliction. For example, for the treatment of tumor-bearing subjects, a “therapeutically effective dosage” preferably inhibits tumor growth by at least about 20%, more preferably by at least about 40%, even more preferably by at least about 60%, and still more preferably by at least about 80% relative to untreated subjects. A therapeutically effective amount of a therapeutic recombinant fusion protein, nucleic acid molecule, expression vector or host cell of the disclosure can decrease tumor size, or otherwise ameliorate symptoms in a subject, which is typically a human or can be another mammal.

The pharmaceutical composition can be a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. See, e.g., Sustained and Controlled Release Drug Delivery Systems, J. R. Robinson, ed., Marcel Dekker, Inc., New York, 1978.

Therapeutic compositions can be administered via medical devices such as (1) needleless hypodermic injection devices (e.g., U.S. Pat. Nos. 5,399,163; 5,383,851; 5,312,335; 5,064,413; 4,941,880; 4,790,824; and 4,596,556); (2) micro-infusion pumps (U.S. Pat. No. 4,487,603); (3) transdermal devices (U.S. Pat. No. 4,486,194); (4) infusion apparatuses (U.S. Pat. Nos. 4,447,233 and 4,447,224); and (5) osmotic devices (U.S. Pat. Nos. 4,439,196 and 4,475,196); the disclosures of which are incorporated herein by reference.

The pharmaceutical composition which may comprise the recombinant fusion protein, nucleic acid molecules, expression vectors or host cells of the present disclosure have numerous in vitro and in vivo utilities involving, for example, treatment of diseases associated with CD40 and/or CD47 signaling such as tumors or infectious diseases.

The disclosure provides a method for treating a disease associated with CD40 signaling and/or CD47 signaling, which may comprise administering to a subject a therapeutically effective amount of the pharmaceutical composition of the present disclosure.

The disease may be a tumor or cancer. The tumor includes, but not limited to leiomyosarcoma, acute lymphocytic leukemia (ALL), acute myeloid leukemia (AML), chronic myeloid leukemia (CML), non-Hodgkin's lymphoma, Hodgkin's lymphoma, chronic lymphocytic leukemia, colon cancer, kidney cancer, prostate cancer, cervix cancer, nasopharynx cancer, breast cancer, osteosarcoma, head and neck squamous cell carcinoma, lung cancer (including small cell lung cancer and non-small cell lung cancer), multiple myeloma, melanoma, hepatocellular carcinoma, liver cancer, cholangiocarcinoma, ovarian cancer, bladder cancer, pancreas cancer (including pancreatic ductal adenocarcinoma), and gastric carcinoma. In certain embodiments, the subject is human.

The disease may be an infectious disease. The infectious disease may be caused by a bacterial, viral or parasitic infection. In certain embodiments, the subject is human.

In yet another aspect, the disclosure provides a method of modulating or enhancing an immune response in a subject comprising administering to the subject the pharmaceutical composition of the disclosure such that the immune response in the subject is modulated/enhanced.

The disclosure also provides a method for reversing or reducing an immune-suppression in a subject in need thereof, comprising administering to the subject the pharmaceutical composition of the disclosure.

Although the present invention and its advantages have been described in detail, it should be understood that various changes, substitutions and alterations can be made herein without departing from the spirit and scope of the invention as defined in the appended claims.

The present disclosure is further illustrated by the following examples, which should not be construed as further limiting. The contents of all figures and all references, Genbank sequences, patents and published patent applications cited throughout this application are expressly incorporated herein by reference.

### EXAMPLES

### Example 1. Construction of Exemplary Recombinant Fusion Proteins

Recombinant fusion proteins were constructed by linking the first Ig-like extracellular domain of human SIRPα isoform 2 (SIRPαV2D1), optionally via a linker, to the N- or C-terminus of the heavy chain, or to the C-terminus of the light chain of a humanized anti-CD40 antibody named as HuCD40-C1H1-V2, as described in WO2021/197335 with a human IgG2 heavy chain constant region. The SIRPαV2D1 was a wild type one, having the amino acid sequence of SEQ ID NO: 11 (X1=V, X2=K, X3=S, X4=K, X5=F), or a variant with certain mutations designed for raising its binding affinity to CD47, having the amino acid sequence of SEQ ID NO: 11 (X1=I, X2=R, X3=T, X4=K, X5=F; or X1=I, X2=R, X3=T, X4=R, X5=V). The anti-CD40 antibody HuCD40-C1H1-V2 was an IgG antibody with two identical heavy chains and two identical light chains, wherein the each heavy chain comprising, from N terminus to C terminus, a heavy chain variable region and a heavy chain constant region, wherein the each light chain comprising, from N terminus to C terminus, a light chain variable region and a light chain constant region, wherein the heavy chain variable region, the light chain variable region, the heavy chain constant region and the light chain constant region respectively comprised the amino acid sequences of SEQ ID NOs: 7, 8, 9 and 10, wherein the heavy chain variable region comprised a VH CDR1, a VH CDR2, and a VH CDR3 having the amino acid sequences of SEQ ID NOs: 1, 2, and 3, respectively, and the light chain variable region comprised a VL CDR1, a VL CDR2 and a VL CDR3 having the amino acid sequences of SEQ ID NOs: 4, 5 and 6, respectively.

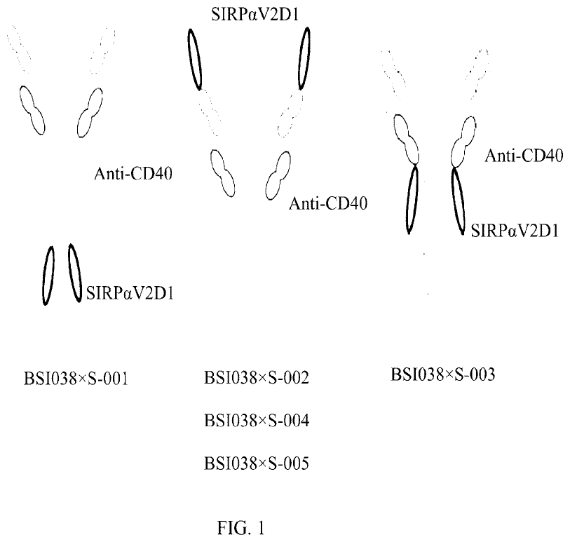

The specific structures and sequences IDs of the exemplary recombinant fusion proteins were set forth in Table 1 below and shown in FIG. 1.

TABLE 1
Molecular Structure and Amino Acid Sequence ID NOs of Exemplary
Recombinant Fusion Proteins

| Fusion | | | |
|---|---|---|---|
| Protein | | | |
| ID | Structure | Constituent polypeptide chains | |
| BSI038×S- | wild-type SIRPαV2D1 | anti-CD40 heavy | anti-CD40 light |
| 001 | linked to C-terminus of | chain-linker- | chain (SEQ ID |
| | each anti-CD40 heavy | SIRPαV2D1 (SEQ | NO: 20) |
| | chain | ID NO: 16) | |
| BSI038×S- | wild-type SIRPαV2D1 | SIRPαV2D1-linker- | anti-CD40 light |
| 002 | linked to N-terminus of | anti-CD40 heavy | chain (SEQ ID |
| | each anti-CD40 heavy | chain (SEQ ID NO: | NO: 20 |
| | chain | 17, X1 = V, X2 = | |
| | | K, X3 = S, X4 = K, | |
| | | X5 = F) | |
| BSI038×S- | wild-type SIRPαV2D1 | anti-CD40 heavy | anti-CD40 light |
| 003 | linked to C-terminus of | chain (SEQ ID NO: | chain-linker- |
| | each anti-CD40 light | 19) | SIRPαV2D1 |
| | chain | | (SEQ ID NO: 18) |
| BSI038×S- | SIRPαV2D1 variant | SIRPαV2D1M1- | anti-CD40 light |
| 004 | linked to N-terminus of | linker-anti-CD40 | chain (SEQ ID |
| | each anti-CD40 heavy | heavy chain (SEQ | NO: 20) |
| | chain | ID NO: 17, X1 = I, | |
| | | X2 = R, X3 = T, | |
| | | X4 = K, X5 = F) | |
| BSI038×S- | SIRPαV2D1 variant | SIRPαV2D1M2- | anti-CD40 light |
| 005 | linked to N-terminus of | linker-anti-CD40 | chain (SEQ ID |
| | each anti-CD40 heavy | heavy chain (SEQ | NO: 20) |
| | chain | ID NO: 17, X1 = I, | |
| | | X2 = R, X3 = T, | |
| | | X4 = R, X5 = V) | |

### Example 2. Expression and Identification of Exemplary Recombinant Fusion Proteins

Briefly, nucleic acids encoding the polypeptide chains constituting each recombinant fusion protein were synthesized, and then inserted into pTT5 vectors, respectively. Afterwards, plasmid DNA extracts of the above vectors were transfected into mammalian cells (CHO cells), and the exemplary recombinant fusion proteins of the disclosure were expressed and secreted by the CHO cells. The recombinant fusion proteins were then purified on the protein A affinity chromatography column, and subject to SDS-PAGE and SEC-HPLC analysis, the results of which were shown in Tables 2-1 and 2-2.

TABLE 2-1
SDS-PAGE Results of Exemplary Recombinant Fusion Proteins

| Sample | Purity (%) |
|---|---|
| BSI038×S-001 | >90% |
| BSI038×S-002 | >90% |
| BSI038×S-003 | >90% |
| BSI038×S-004 | >90% |
| BSI038×S-005 | >90% |

TABLE 2-2
SEC-HPLC Results of Exemplary Recombinant Fusion Proteins

| Sample | Monomer (%) |
|---|---|
| BSI038×S-001 | 98.76 |
| BSI038×S-002 | 99.70 |
| BSI038×S-003 | 99.53 |
| BSI038×S-004 | 98.94 |
| BSI038×S-005 | 95.89 |

It can be seen from the results that the recombinant fusion proteins of the disclosure exhibited high purity, and most fusion proteins were present as monomers.

### Example 3. Binding Affinity Determination of Exemplary Recombinant Fusion Proteins Using BIACORE Surface Plasmon Resonance Technology

The purified recombinant fusion proteins were characterized for their binding affinity and binding kinetics by Biacore T200 system (GE healthcare, Pittsburgh, PA, USA). The anti-CD40 antibody HuCD40-C1H1-V2 (having the heavy chain and the light chain of SEQ ID NOs: 19 and 20), Selicrelumab (an agonistic anti-CD40 antibody, Roche Inc., prepared in-house with heavy and light chains of SEQ ID NOs: 27 and 28), and SIRPα-Fc fusion proteins, including SIRPαV2D1M1-Fc (IgG2) (SEQ ID NO: 21, X1=I, X2=R, X3=T, X4=K, X5=F), SIRPαV2D1M2-Fc (IgG2) (SEQ ID NO: 21, X1=I, X2=R, X3=T, X4=R, X5=V), SIRPα-isoform2-Fc (SEQ ID NO: 25) were used as positive controls.

A Protein A chip (Cat #: 29-1275-56, GE healthcare) was used for affinity determination. The recombinant fusion proteins of the disclosure and the positive controls each at the concentration of 2 μg/ml were respectively flowed onto the chip at a flow rate of 10 μl/min. Then, serially diluted recombinant human CD40-his proteins (Cat #: CD0-H5228, Acro biosystems), human CD47-his proteins (Cat #: CD7-H5227, Acro biosystems), cynomolgus CD40-his proteins (Biosion in house made, SEQ ID NO: 29) or cynomolgus CD47-his proteins (Cat #: CD7-C52H1, Acro biosystems), 2-fold dilution in HBS-EP⁺ buffer (provided by Biacore) with a starting concentration of 200 nM, were flowed onto the chip at a flow rate of 30 μl/min. The association kinetics was followed for 2 minutes and the dissociation kinetics was followed for 10 minutes. The association and dissociation curves were fit to a 1:1 Langmuir binding model using the Biacore evaluation software. The K_{D}, Kₐ and Kₐ values were determined and summarized in Tables 3-1, 3-2, 3-3, and 3-4 below.

TABLE 3-1
Binding Affinity of Recombinant Fusion Proteins to Human CD40

| | Kinetics on Biacore | | |
|---|---|---|---|
| | Human CD40-his | | |
| | Kₐ | K_{d} | K_{D} |
| Sample | (M⁻¹s⁻¹) | (s⁻¹) | (M) |
| BSI038×S-001 | 2.44E+06 | 1.88E−03 | 7.71E−10 |
| BSI038×S-002 | 2.53E+06 | 1.84E−03 | 7.29E−10 |
| BSI038×S-003 | 2.57E+06 | 1.83E−03 | 7.10E−10 |
| HuCD40-C1H1-V2 | 1.62E+06 | 1.69E−03 | 1.05E−09 |
| BSI038×S-004 | 1.75E+06 | 0.001639 | 9.37E−10 |
| BSI038×S-005 | 1.77E+06 | 0.001654 | 9.35E−10 |
| Selicrelumab | 3.60E+05 | 0.002308 | 6.41E−09 |

TABLE 3-2
Binding Affinity of Recombinant Fusion Proteins to Human CD47

| | Kinetics on Biacore | | |
|---|---|---|---|
| | Human CD47-his | | |
| | Kₐ | K_{d} | K_{D} |
| Sample | (M⁻¹s⁻¹) | (s⁻¹) | (M) |
| BSI038×S-001 | | BDL | |
| BSI038×S-002 | 1.09E+06 | 6.86E−03 | 6.31E−09 |
| BSI038×S-003 | | BDL | |
| SIRPα-isoform2-Fc | 1.66E+06 | 9.18E−03 | 5.52E−09 |
| BSI038×S-004 | 5.94E+05 | 5.86E−04 | 9.87E−10 |
| BSI038×S-005 | 5.18E+05 | 5.98E−04 | 1.15E−09 |
| SIRPαV2D1M1-Fc(IgG2) | 4.21E+05 | 4.01E−04 | 9.52E−10 |
| SIRPαV2D1M2-Fc(IgG2) | 3.88E+05 | 4.17E−04 | 1.07E−09 |

BDL: below detection limit

TABLE 3-3
Binding Affinity of Recombinant Fusion Proteins to Cyno CD40

| | Kinetics on Biacore | | |
|---|---|---|---|
| | Cyno CD40-his | | |
| | Kₐ | K_{d} | K_{D} |
| Sample | (M⁻¹s⁻¹) | (s⁻¹) | (M) |
| BSI038×S-004 | 7.53E+05 | 0.00163 | 2.16E−09 |
| BSI038×S-005 | 7.59E+05 | 0.001672 | 2.20E−09 |
| Selicrelumab | 1.31E+06 | 0.002192 | 1.68E−08 |

TABLE 3-4
Binding Affinity of Recombinant Fusion Proteins to Cyno CD47

| | Kinetics on Biacore | | |
|---|---|---|---|
| | Cyno CD47-his | | |
| | Kₐ | K_{d} | K_{D} |
| Sample | (M⁻¹s⁻¹) | (s⁻¹) | (M) |
| BSI038×S-004 | 9.48E+04 | 5.76E−04 | 6.08E−09 |
| BSI038×S-005 | 9.18E+04 | 4.95E−04 | 5.39E−09 |
| SIRPαV2M1-Fc(IgG2) | 7.60E+04 | 3.71E−04 | 4.88E−09 |
| SIRPαV2M2-Fc(IgG2) | 6.24E+04 | 4.38E−04 | 7.01E−09 |

All the recombinant fusion proteins of the disclosure specifically bound to human CD40 and cyno CD40 with high binding affinity.

Further, the recombinant fusion proteins of the disclosure also specifically bound to human CD47 and cyno CD47, wherein BSI038×S-004 and BSI038×S-005's CD47 binding affinity was comparable to that of SIRPαV2D1M1-Fc (IgG2) and SIRPαV2D1M2-Fc (IgG2), and better than that of BSI038×S-002.

### Example 4. Binding Activity of Exemplary Recombinant Fusion Proteins

The binding activity of the recombinant fusion proteins of the disclosure to human CD40 were further determined by Flow Cytometry (FACS), using Biosion in-house prepared 293T-CD40 cells stably expressing full length human CD40 (uniprot #P25942-1). The 293T-CD40 cells were prepared by transfecting 293T cells with pCMV-T-P plasmids inserted with DNA coding human CD40 (uniprot #P25942-1) between EcoRI and XbaI sites, following the instruction of lipofectamine 3000 transfection reagent (Thermo Fisher).

Briefly, the 293T-CD40 cells were harvested from cell culture flasks, washed twice and resuspended in phosphate buffered saline (PBS) containing 2% v/v Fetal Bovine Serum (FACS buffer). Then, 1×10⁵ 293T-CD40 cells in each well of the 96 well-plates were incubated with 100 μl serially diluted recombinant fusion proteins of the disclosure or controls (starting from 10 μg/mL with a 5-fold serial dilution in FACS buffer) for 40 minutes on ice. Cells were washed twice with FACS buffer, and Goat Anti-Human IgG (Fab)-PE (1:1000 dilution in FACS buffer, Cat #: 109-116-097, Jackson ImmunoResearch) was added, 100 μl/well. Following an incubation of 40 minutes at 4° C. in dark, cells were washed three times and resuspended in FACS buffer. Fluorescence was measured using a Becton Dickinson FACS Canto II-HTS equipment and plotted against the recombinant fusion protein or controls concentration. Data was analyzed using Graphpad Prism and EC₅₀ values were reported. The results were shown in FIG. 2.

The binding activity of the recombinant fusion proteins to cell-surface human CD47 was also tested by flow cytometry (FACS), using Biosion in-house prepared 293F-CD47 cells expressing full length human CD47 (NCBI #NP_942088.1) on cell membranes. The 293F-CD47 cells were prepared by transfecting 293F cells with pCMV-T-P plasmids inserted with the DNA coding human CD47 (NP_942088.1) between EcoRI and XbaI sites, following the instruction of lipofectamine 3000 transfection reagent (Thermo Fisher).

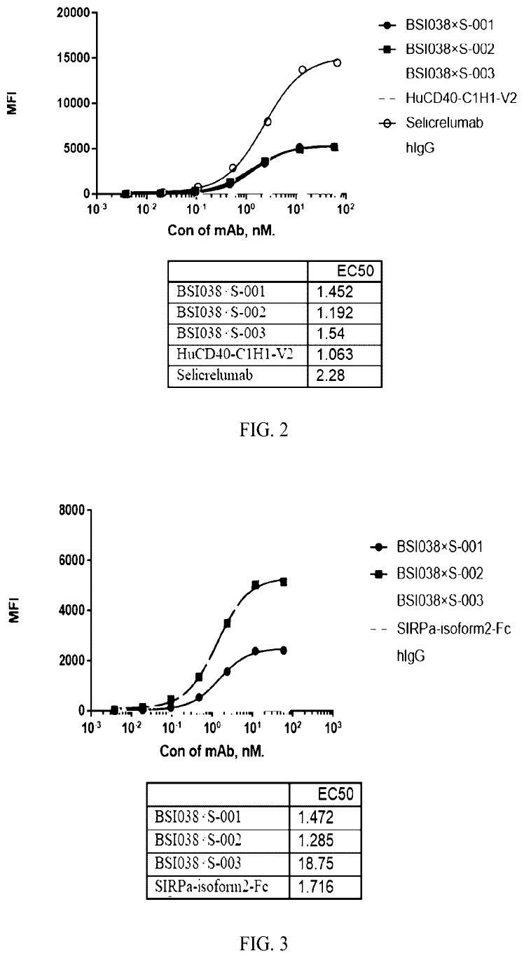

The 293F-CD47 cells were harvested from cell culture flasks, washed twice and resuspended in phosphate buffered saline (PBS) containing 2% v/v Fetal Bovine Serum (FACS buffer). Then, 1×10⁵ 293F-CD47 cells in each well of the 96 well-plates were incubated with 100 μl serially diluted recombinant fusion proteins of the disclosure or controls (starting from 10 g/mL, 5-fold serial dilution in FACS buffer) for 40 minutes on ice. Cells were washed twice with FACS buffer, and Goat Anti-Human IgG (Fc)-PE (1:1000 dilution in FACS buffer, Cat #: 109-115-098, Jackson ImmunoResearch) was added, 100 μl/well. Following an incubation of 50 minutes at 4° C. in dark, cells were washed three times and resuspended in FACS buffer. Fluorescence was measured using a Becton Dickinson FACS Canto II-HTS equipment, and the MFI (mean fluorescence intensity) was plotted against the recombinant fusion protein or controls concentration. Data was analyzed using Graphpad Prism and EC₅₀ values were reported. The results were shown in FIG. 3.

It can be seen from FIG. 2 that the recombinant fusion proteins of the disclosure specifically bound to human CD40.

According to FIG. 3, the recombinant fusion proteins of the disclosure specifically bound to human CD47. Particularly the binding activity of BSI038×S-002 to human CD47 was comparable to that of the positive control SIRPα-isoform2-Fc.

The binding activity of the recombinant fusion proteins to human CD40 and CD47 was also tested by dual-binding ELISA assay. Briefly, 100 μl/well human CD40-Fc proteins (Cat #: CD0-H5253, ACRO Biosystems) were coated on 96-well plates at 0.2 μg/mL in PBS and incubated overnight at 2-8° C. The next day, the plates were washed 3 times using wash buffer (PBS+0.05% v/v Tween-20, PBST), and then blocked with block buffer (PBS+1% w/v BSA) for 3 hours at 37° C. The plates were then washed 3 times using the wash buffer.

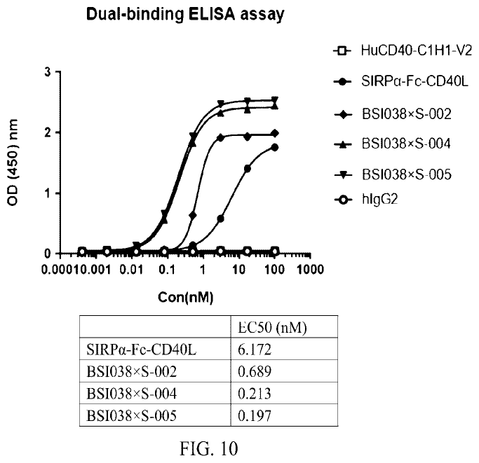

Serially diluted recombinant fusion proteins of the disclosure or controls (starting at 100 nM with a 6-fold serial dilution) in block buffer were added to the CD40-Fc bound plates, 100 μL per well, and incubated at 37° C. for 1 hour. The plates were washed 3 times using wash buffer, and then added and incubated for 1 hour at 37° C. with 100 μl/well 3 μg/mL human CD47-his protein (Cat #: CD7-H5227, ACRO Biosystems). The plates were washed again using wash buffer, added with 100 μl/well Anti-His tag Antibody (HRP) (1:2000 dilution in block buffer, Cat #: 105327-MM02T-H, Sino Biological) and incubated for 1 hour at 37° C. The plates were washed again using wash buffer. Finally, the plates were revealed with the addition of TMB and the reaction was stopped by adding 1M H₂SO₄. The absorbance was read using a microplate reader set to 450 nm, then the OD (450) values were plotted against the recombinant fusion protein or controls concentration. Data was analyzed using Graphpad Prism and EC₅₀ values were reported. The results were shown in FIG. 10.

It can be seen from FIG. 10 that the recombinant fusion proteins of the disclosure specifically bound to human CD40 and CD47, and exhibited higher binding activity than SIRPα-Fc-CD40L.

### Example 5. Blocking Activity of Exemplary Recombinant Fusion Proteins on CD40-CD40L or CD47-SIRPα Binding

### 5.1 Inhibitory Effect on CD40-CD40L Binding Tested in Ligand Blocking ELISA

The ability of the recombinant fusion proteins of the disclosure to block CD40-CD40L binding was measured in a competitive ELISA assay. Briefly, 100 μl/well human CD40-Fc proteins (prepared in-house with amino acid sequence of SEQ ID NO: 22) were coated on 96-well micro plates at 2 μg/mL in coating buffer (carbonate/bicarbonate buffer) and incubated overnight at 4° C. The next day, the plates were washed once with wash buffer (PBS+0.05% v/v Tween-20, PBST), and then blocked with 5% w/v non-fatty milk in PBST for 2 hours at 37° C. The plates were then washed 4 times using the wash buffer.

Serially diluted recombinant fusion proteins of the disclosure or controls (starting at 10 g/mL with a 5-fold serial dilution) in PBST with 2.5% w/v non-fatty milk were added to the CD40-Fc bound plates, 100 μl per well, and incubated at 37° C. for 40 minutes. The plates were washed 4 times using wash buffer, and then added and incubated for 40 minutes at 37° C. with 100 μl/well 95 ng/mL biotin-labeled human CD40L-his protein (Cat #: 10239-H08E, Sino biological). The plates were washed again using wash buffer, added with 100 μl/well streptavidin conjugated HRP (1:10000 dilution in PBST buffer, Cat #: 016-030-084, Jackson ImmunoResearch) and incubated for 40 minutes at 37° C. The plates were washed again using wash buffer. Finally, the plates were revealed with the addition of TMB and the reaction was stopped by adding 1M H₂SO₄. The absorbance was read on a microplate reader using the dual wavelength mode with 450 nm for TMB and 630 nm as the reference wavelength, then the OD (450-630) values were plotted against the recombinant fusion protein or controls concentration. Data was analyzed using Graphpad Prism and IC₅₀ values were reported. The results were shown in FIG. 4.

### 5.2 Inhibitory Effect on CD47-CD172a Binding Tested in Ligand Blocking ELISA

The ability of the recombinant fusion proteins of the disclosure to block CD47-CD172a binding was measured in a competitive ELISA assay. Briefly, 100 μl/well human CD172a-Fc proteins (prepared in-house with amino acid sequence of SEQ ID NO: 24) were coated on 96-well micro plates at 2 μg/mL in coating buffer (carbonate/bicarbonate buffer) and incubated overnight at 4° C. The next day, the plates were washed once with wash buffer (PBS+0.05% v/v Tween-20, PBST), and then blocked with 5% w/v non-fatty milk in PBST for 2 hours at 37° C. The plates were then washed 4 times using wash buffer.

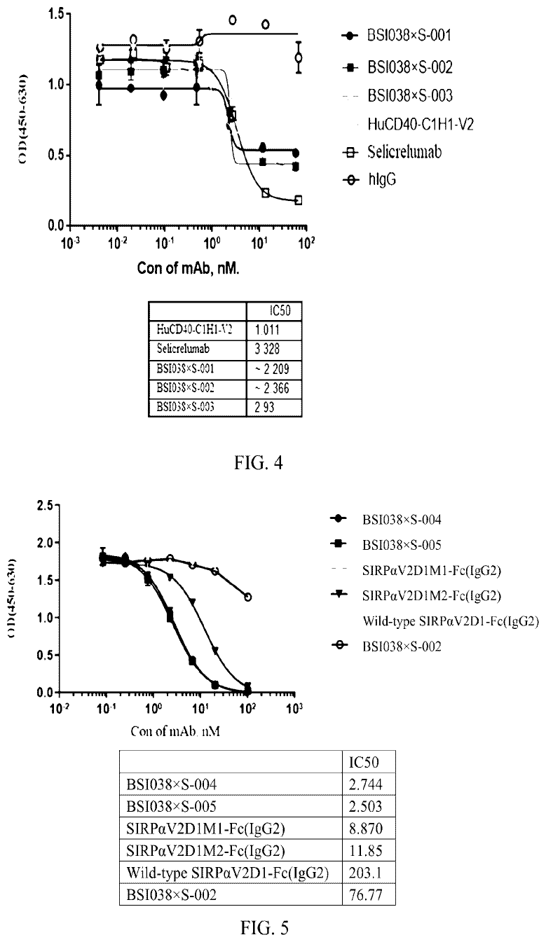

Serially diluted recombinant fusion proteins of the disclosure or controls (starting at 100 nM with a 5-fold serial dilution) were mixed with 20 ng/ml biotin-labeled human CD47-Fc (Biosion in house made, SEQ ID NO: 23) in PBST with 2.5% w/v non-fatty milk. The mixtures were incubated at 37° C. for 40 minutes, and respectively added to the CD172a-Fc bound plates, 100 μl per well. The plates were incubated at 37° C. for 40 minutes, washed 4 times using wash buffer, added with 100 μl/well streptavidin conjugated HRP (1:10000 dilution in PBST buffer, Cat #: 016-030-084, Jackson ImmunoResearch), incubated for 40 minutes at 37° C., and washed again using wash buffer. Finally, the plates were revealed with the addition of TMB and the reaction was stopped by adding 1M H₂SO₄. The plates were read on a microplate reader using the dual wavelength mode with 450 nm for TMB and 630 nm as the reference wavelength, and the OD (450-630) values were plotted against the recombinant fusion protein or controls concentration. Data was analyzed using Graphpad Prism and IC₅₀ values were reported. Wild-type SIRPαV2D1-Fc (IgG2) (SEQ ID NO: 21, X1=V, X2=K, X3=S, X4=K, X5=F) was used as a positive control. The results were shown in FIG. 5.

### 5.3 Inhibitory Effect on CD47-SIRPα Binding Tested in Cell-Based Ligand Blocking FACS

The activity of the recombinant fusion proteins of the disclosure to block SIRPα binding to cell-surface CD47 was evaluated by Flow Cytometry (FACS), using in-house made 293F-CD47 cells.

Briefly, the 293F-CD47 cells were prepared by transfecting the 293F cells with pCMV-T-P plasmid inserted with the DNA coding human CD47 (NP_942088.1) between EcoRI and XbaI sites, following the instruction of lipofectamine 3000 transfection reagent (Thermo Fisher).

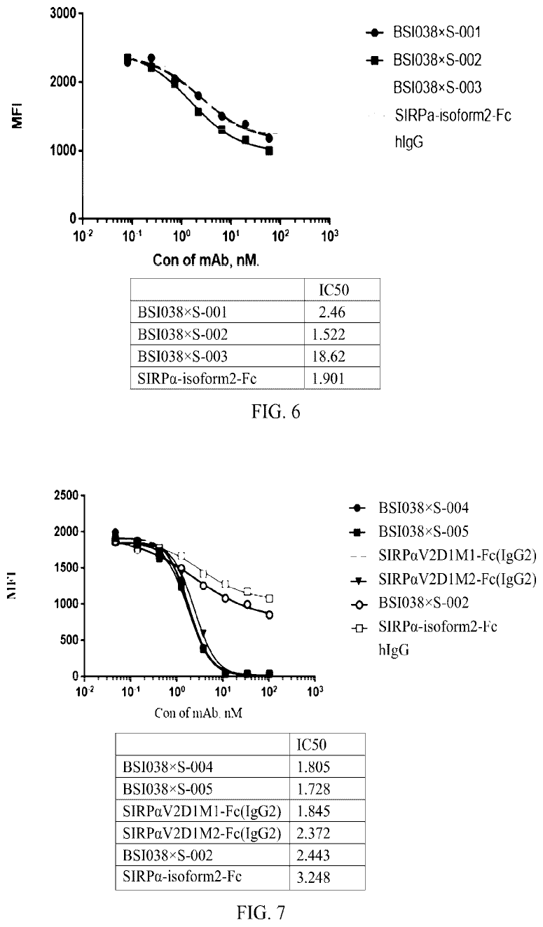

The recombinant fusion proteins of the disclosure or controls were diluted in PBS containing 2% v/v Fetal Bovine Serum (FACS buffer), with a 3-fold serial dilution starting from 100 nM. Meanwhile, the 293F-CD47 cells in log growth stage were harvested, washed twice using FACS buffer, centrifuged and collected. Then 1×10⁵ cells were suspended and incubated in 100 μl of the diluted antibodies or controls in each well of the 96-well plates for 60 minutes at 4° C. The plates were washed twice and then incubated with 100 μL/well 148 ng/mL biotin-labeled human CD172a-Fc proteins (prepared in-house with amino acid sequence of SEQ ID NO: 24) in FACS buffer for 60 minutes at 4° C. The plates were washed twice with FACS buffer, and then added and incubated for 40 minutes at 4° C. in dark with 100 μl/well R-Phycoerythrin Streptavidin (1:500 dilution in FACS buffer, Cat #: 016-110-084, Jackson ImmunoResearch). The cells were washed twice and resuspended in FACS buffer. Fluorescence was measured using a Becton Dickinson FACS Canto II-HTS equipment, and the MFI (mean fluorescence intensity) was plotted against the recombinant fusion protein or controls concentration. Data was analyzed using Graphpad Prism and IC₅₀ values were reported. The results were shown in FIGS. 6 and 7.

It can be seen from FIG. 4 that the recombinant fusion proteins of the disclosure were capable of blocking human CD40 binding to human CD40L, with comparable activity to the positive controls.

It can be seen from FIG. 5 that BSI038×S-002 was capable of blocking human CD47 binding to human CD172a, with higher activity than Wild-type SIRPαV2D1-Fc (IgG2). Further, BSI038×S-004 and BSI038×S-005 were capable of blocking human CD47 binding to human CD172a, with higher activity than the positive controls SIRPαV2D1M1-Fc (IgG2) and SIRPαV2D1M2-Fc (IgG2).

Further, according to FIGS. 6 and 7, the recombinant fusion proteins of the disclosure were capable of blocking binding of human SIRPα to cell-surface human CD47, with most having comparable blocking activity compared to the positive controls. Further, BSI038×S-004 and BSI038×S-005 showed higher CD47-SIRPα blocking activity than BSI038×S-002.

### Example 6. Cell Based Functional Assay of Exemplary Recombinant Fusion Proteins

### 6.1 CD40 Agonistic Activity Tested in Cell Based Reporter Assay

The recombinant fusion proteins of the disclosure were further tested for their agonistic activity on CD40 signaling using a CD40-expressing reporter cell line 293T-NF-κB-Luc-CD40 which stably expressed full length human CD40 (uniprot No. P25942-1). The 293T-NF-κB-Luc-CD40 cells were prepared, following the instruction of lipofectamine 3000 transfection reagent (Thermo Fisher), by transfecting 293T cells with pGL4.32 [luc2P/NF-κB-RE/Hygro]vectors (Promega, GenBank® Accession Number: EU581860) and later pCMV-T-P plasmids inserted with the DNA coding human CD40 between EcoRI and XbaI sites. When CD40 agonists were contacted with these cells, the CD40 signaling would be activated and the luciferase expression, which can be measured in a luminescence assay, would be up-regulated by the NF-κB transcription factor. A recombination protein, SIRPα-Fc-CD40L which has been described in the reference “K, Patel A, et al. CD40 Enhances Type I Interferon Responses Downstream of CD47 Blockade, Bridging Innate and Adaptive Immunity. Cancer Immunol Res. 2020 February; 8 (2): 230-245”, having the amino acid sequence of SEQ ID NO: 26, was used as the control.

Briefly, 5×10³ 293T-NF-κB-Luc-CD40 cells at the log growth phase in 20 μL DMEM medium (Cat #: 10566-016, Gibco) supplemented with 10% FBS (Cat #: 10099-141, Gibco) were plated into each well of the 384-well cell culture plates (Cat #: 3707, Corning). The plates were added with 20 μl/well serially diluted recombinant fusion proteins of the disclosure or controls (starting from 200 nM, 3-fold serial dilution in the culture medium), and incubated at 37° C. for 6 hours. Then, the plates were added with the reagents of ONE-Glo™ Luciferase Assay System (30 μl/well, Cat #: E6120, Promega) and incubated for 5 minutes at room temperature. Chemiluminescence was measured using a Tecan Infinite® 200 Pro equipment. Data was analyzed using Graphpad Prism and EC₅₀ values were reported.

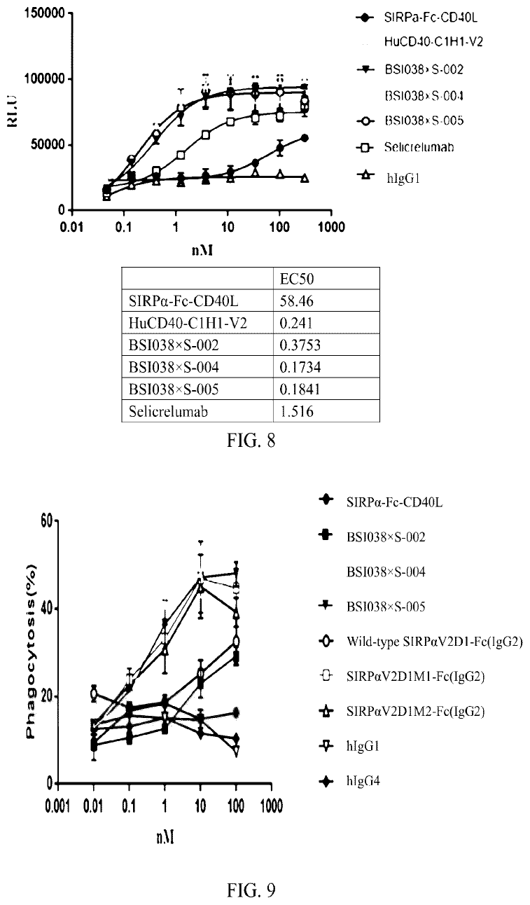

The results were shown in FIG. 8.

It can be seen that BSI038×S-002, BSI038×S-004 and BSI038×S-005 showed CD40 agonistic activity, which was comparable to that of HuCD40-C1H1-V2 and much higher than that of Selicrelumab and SIRPα-Fc-CD40L.

### 6.2 Capability to Induce Phagocytosis of Cancer Cells

The recombinant fusion proteins of the disclosure were further tested for their capability to induce phagocytosis of cancer cells, using the recombinant proteins SIRPα-isoform2-Fc, SIRPαV2D1M1-Fc (IgG2), SIRPαV2D1M2-Fc (IgG2), SIRPα-Fc-CD40L and Wild-type SIRPαV2D1-Fc (IgG2) as the controls.

Briefly, monocytes were isolated from frozen human PBMCs with EasySep™ Human Monocyte Enrichment Kit without CD16 Depletion (Cat #: 19058, Stemcell) according to the user manual, and seeded on 6-well plates in RPMI1640+10% FBS+1% Penicillin Streptomycin+75 ng/ml human M-CSF for 6 days, with fresh cell culture media added on Day 3. The macrophages were detached from the plates and re-seeded on 96-well plates on Day 6 and cultured overnight.

CFSE-labeled Jurkat cells were harvested, and incubated at a density of 0.4 million/ml in 50 μl serially diluted recombinant fusion proteins of the disclosure or controls at room temperature for 30 min. Then, the Jurkat cell/antibody mixtures were added to the macrophages as collected above at an effect cell: target cell ratio of 1:2, and co-cultured at 37° C. for 4 hours.

All cells were collected from the plates with the Accutase® cell detachment solution and washed once with FACS buffer. Afterwards, cells were blocked with Human TruStain FcX™ Fc Receptor Blocking Solution (Cat #: 422302, Biolegend), and then stained with anti-human CD11b APC (Cat #: 301310, Biolegend). Phagocytosis percentage (%) was determined as the ratio of CFSE⁺ population to CD11b⁺ population (number of CD11b⁺ macrophages containing the engulfed CFSE⁺ cells/total number of counted CD11b⁺ macrophages) by Flow cytometry.

The result was shown in FIG. 9.

It can be seen that BSI038×S-002, BSI038×S-004 and BSI038×S-005 were able to induce phagocytosis of tumor cells by macrophages, and exhibited more potent activity than SIRPα-Fc-CD40L.

### Example 7. Thermal Stability of Exemplary Recombinant Fusion Proteins

The recombinant fusion proteins were tested for their thermal stability. Briefly, a protein thermal shift assay was used to determine Tm (melting temperature) using a GloMelt™ Thermal Shift Protein Stability Kit (Cat #: 33022-T, Biotium). Briefly, the GloMelt™ dye was allowed to thaw and reach room temperature. The vial containing the dye was vortexed and centrifuged. Then, 10× dye was prepared by adding 5 μL 200× dye to 95 μL PBS. Then, 2 μL 10× dye and 10 μg recombinant fusion proteins of the disclosure or controls were added, and PBS was added to a total reaction volume of 20 μL. The tubes containing the dye and fusion proteins or controls were briefly spun and placed in real-time PCR thermocycler (Roche, LightCycler 480 II) set up with a melt curve program having the parameters in Table 4.

TABLE 4
Parameters for Melt Curve Program

| Profile step | Temperature | Ramp rate | Holding Time |
|---|---|---|---|
| Initial hold | 25° C. | NA | 30 s |
| Melt curve | 25-99° C. | 0.1° C./s | NA |

TABLE 5
Melting temperatures of recombinant fusion proteins

| | Tm (melting temperature) ° C. | |
|---|---|---|
| Sample | Tm1 | Tm2 |
| BSI038×S-001 | 61.0 | 69.5 |
| BSI038×S-002 | 64.5 | NA |
| BSI038×S-003 | 61.0 | 77.5 |
| BSI038×S-004 | 64.0 | NA |
| BSI038×S-005 | 64.0 | NA |
| HuCD40-C1H1-V2 | 65.0 | NA |
| SIRPα-isoform2-Fc | 65.5 | NA |

The results were shown in Table 5, suggesting that the recombinant fusion proteins were probably stable in human body.

While the disclosure has been described above in connection with one or more embodiments, it should be understood that the disclosure is not limited to those embodiments, and the description is intended to cover all alternatives, modifications, and equivalents, as may be included within the spirit and scope of the appended claims. All referenced cited herein are further incorporated by reference in their entirety.

Sequences in the present application are summarized below.

Description/
Sequence/SEQ ID NO.

VH CDR1 of HuCD40-C1H1-V2
NYGIS (SEQ ID NO: 1)
VH CDR2 of HuCD40-C1H1-V2
SISSGGDNTYYPDNVKG (SEQ ID NO: 2)
VH CDR3 of HuCD40-C1H1-V2
AGEKAMDY (SEQ ID NO: 3)
VL CDR1 of HuCD40-C1H1-V2
RASQTINNNLH (SEQ ID NO: 4)
VL CDR2 of HuCD40-C1H1-V2
YASQSIS (SEQ ID NO: 5)
VL CDR3 of HuCD40-C1H1-V2
QQFSSWPLT (SEQ ID NO: 6)
VH of HuCD40-C1H1-V2
EVQLVESGGGLVKPGGSLRLSCAASGFTFSNYGISWVRQAPGKGLEWVSS
ISSGGDNTYYPDNVKGRFTISRDNAKNSLYLQMNSLRAEDTAVYYCARAG
EKAMDYWGQGTLVTVSS (SEQ ID NO: 7)
VL of HuCD40-C1H1-V2
EIVLTQSPATLSLSPGERATLSCRASQTINNNLHWYQQKPGQAPRLLIKY
ASQSISGIPARFSGSGSGTDFTLTISSLEPEDFAVYFCQQFSSWPLTFGG
GTKVEIK (SEQ ID NO: 8)
Heavy chain constant region
ASTKGPSVFPLAPCSRSTSESTAALGCLVKDYFPEPVTVSWNSGALTSGV
HTFPAVLQSSGLYSLSSVVTVPSSNFGTQTYTCNVDHKPSNTKVDKTVER
KCCVECPPCPAPPVAGPSVFLFPPKPKDTLMISRTPEVTCVVVDVSHEDP
EVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDIAVEWESNGQPENNYKTTPPMLDSDGSFFLYSKLTVDKSRWQQGN
VFSCSVMHEALHNHYTQKSLSLSPGK (SEQ ID NO: 9)
Light chain constant region
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC (SEQ ID NO: 10)
Wild-type first Ig-like extracellular domain of
human SIRPα-isoform2 (Wild-type SIRPαV2D1)
EEELQVIQPDKSVSVAAGESAILHCTX1TSLIPVGPIQWFRGAGPARELI
YNQX2EGHFPRVTTVSEX3TX4RENMDFSISISNITPADAGTYYCVKFRK
GSPDTEX5KSGAGTELSVRAKPSAPVVSGPAARATP (SEQ ID NO:
11) X1 = V, X2 = K, X3 = S, X4 = K, X5 = F
EEELQVIQPDKSVSVAAGESAILHCTVTSLIPVGPIQWFRGAGPARELIY
NQKEGHFPRVTTVSESTKRENMDFSISISNITPADAGTYYCVKFRKGSPD
TEFKSGAGTELSVRAKPSAPVVSGPAARATP
SIRPαV2D1M1
SEQ ID NO: 11, X1 = I, X2 = R, X3 = T, X4 = K,
X5 = F
EEELQVIQPDKSVSVAAGESAILHCTITSLIPVGPIQWFRGAGPARELIY
NQREGHFPRVTTVSETTKRENMDFSISISNITPADAGTYYCVKFRKGSPD
TEFKSGAGTELSVRAKPSAPVVSGPAARATP
SIRPαV2D1M2
SEQ ID NO: 11, X1 = I, X2 = R, X3 = T, X4 = R,
X5 = V
EEELQVIQPDKSVSVAAGESAILHCTITSLIPVGPIQWFRGAGPARELIY
NQREGHFPRVTTVSETTRRENMDFSISISNITPADAGTYYCVKFRKGSPD
TEVKSGAGTELSVRAKPSAPVVSGPAARATP
Linker
GGGGS (SEQ ID NO: 12)
GGGGSGGGGS (SEQ ID NO: 13)
GGGGSGGGGSGGGGS (SEQ ID NO: 14)
GGGGSGGGGSGGGGSGGGGS (SEQ ID NO: 15)
anti-CD40 heavy chain-linker-SIRPαV2D1
EVQLVESGGGLVKPGGSLRLSCAASGFTFSNYGISWVRQAPGKGLEWVSS
ISSGGDNTYYPDNVKGRFTISRDNAKNSLYLQMNSLRAEDTAVYYCARAG
EKAMDYWGQGTLVTVSSASTKGPSVFPLAPCSRSTSESTAALGCLVKDYF
PEPVTVSWNSGALTSGVHTFPAVLQSSGLYSLSSVVTVPSSNFGTQTYTC
NVDHKPSNTKVDKTVERKCCVECPPCPAPPVAGPSVFLFPPKPKDTLMIS
RTPEVTCVVVDVSHEDPEVQFNWYVDGVEVHNAKTKPREEQFNSTFRVVS
VLTVVHQDWLNGKEYKCKVSNKGLPAPIEKTISKTKGQPREPQVYTLPPS
REEMTKNQVSLTCLVKGFYPSDIAVEWESNGQPENNYKTTPPMLDSDGSF
FLYSKLTVDKSRWQQGNVFSCSVMHEALHNHYTQKSLSLSPGKGGGGSEE
ELQVIQPDKSVSVAAGESAILHCTVTSLIPVGPIQWFRGAGPARELIYNQ
KEGHFPRVTTVSESTKRENMDFSISISNITPADAGTYYCVKFRKGSPDTE
FKSGAGTELSVRAKPSAPVVSGPAARATP* (SEQ ID NO: 16)
Wild-type SIRPαV2D1-linker-anti-CD40 heavy chain
EEELQVIQPDKSVSVAAGESAILHCTX1TSLIPVGPIQWFRGAGPARELI
YNQX2EGHFPRVTTVSEX3TX4RENMDFSISISNITPADAGTYYCVKFRK
GSPDTEX5KSGAGTELSVRAKPSAPVVSGPAARATPGGGGSGGGGSEVQL
VESGGGLVKPGGSLRLSCAASGFTFSNYGISWVRQAPGKGLEWVSSISSG
GDNTYYPDNVKGRFTISRDNAKNSLYLQMNSLRAEDTAVYYCARAGEKAM
DYWGQGTLVTVSSASTKGPSVFPLAPCSRSTSESTAALGCLVKDYFPEPV
TVSWNSGALTSGVHTFPAVLQSSGLYSLSSVVTVPSSNFGTQTYTCNVDH
KPSNTKVDKTVERKCCVECPPCPAPPVAGPSVFLFPPKPKDTLMISRTPE
VTCVVVDVSHEDPEVQFNWYVDGVEVHNAKTKPREEQFNSTFRVVSVLTV
VHQDWLNGKEYKCKVSNKGLPAPIEKTISKTKGQPREPQVYTLPPSREEM
TKNQVSLTCLVKGFYPSDIAVEWESNGQPENNYKTTPPMLDSDGSFFLYS
KLTVDKSRWQQGNVFSCSVMHEALHNHYTQKSLSLSPGK*(SEQ ID
NO: 17) X1 = V, X2 = K, X3 = S, X4 = K, X5 = F
EEELQVIQPDKSVSVAAGESAILHCTVTSLIPVGPIQWFRGAGPARELIY
NQKEGHFPRVTTVSESTKRENMDFSISISNITPADAGTYYCVKFRKGSPD
TEFKSGAGTELSVRAKPSAPVVSGPAARATPGGGGSGGGGSEVQLVESGG
GLVKPGGSLRLSCAASGFTFSNYGISWVRQAPGKGLEWVSSISSGGDNTY
YPDNVKGRFTISRDNAKNSLYLQMNSLRAEDTAVYYCARAGEKAMDYWGQ
GTLVTVSSASTKGPSVFPLAPCSRSTSESTAALGCLVKDYFPEPVTVSWN
SGALTSGVHTFPAVLQSSGLYSLSSVVTVPSSNFGTQTYTCNVDHKPSNT
KVDKTVERKCCVECPPCPAPPVAGPSVFLFPPKPKDTLMISRTPEVTCVV
VDVSHEDPEVQFNWYVDGVEVHNAKTKPREEQFNSTFRVVSVLTVVHQDW
LNGKEYKCKVSNKGLPAPIEKTISKTKGQPREPQVYTLPPSREEMTKNQV
SLTCLVKGFYPSDIAVEWESNGQPENNYKTTPPMLDSDGSFFLYSKLTVD
KSRWQQGNVFSCSVMHEALHNHYTQKSLSLSPGK
SIRPαV2D1M1-linker-anti-CD40 heavy chain
SEQ ID NO: 17, X1 = I, X2 = R, X3 = T, X4 = K,
X5 = F
EEELQVIQPDKSVSVAAGESAILHCTITSLIPVGPIQWFRGAGPARELIY
NQREGHFPRVTTVSETTKRENMDFSISISNITPADAGTYYCVKFRKGSPD
TEFKSGAGTELSVRAKPSAPVVSGPAARATPGGGGSGGGGSEVQLVESGG
GLVKPGGSLRLSCAASGFTFSNYGISWVRQAPGKGLEWVSSISSGGDNTY
YPDNVKGRFTISRDNAKNSLYLQMNSLRAEDTAVYYCARAGEKAMDYWGQ
GTLVTVSSASTKGPSVFPLAPCSRSTSESTAALGCLVKDYFPEPVTVSWN
SGALTSGVHTFPAVLQSSGLYSLSSVVTVPSSNFGTQTYTCNVDHKPSNT
KVDKTVERKCCVECPPCPAPPVAGPSVFLFPPKPKDTLMISRTPEVTCVV
VDVSHEDPEVQFNWYVDGVEVHNAKTKPREEQFNSTFRVVSVLTVVHQDW
LNGKEYKCKVSNKGLPAPIEKTISKTKGQPREPQVYTLPPSREEMTKNQV
SLTCLVKGFYPSDIAVEWESNGQPENNYKTTPPMLDSDGSFFLYSKLTVD
KSRWQQGNVFSCSVMHEALHNHYTQKSLSLSPGK*
SIRPαV2D1M2-linker-anti-CD40 heavy chain
SEQ ID NO: 17, X1 = I, X2 = R, X3 = T, X4 = R,
X5 = V
EEELQVIQPDKSVSVAAGESAILHCTITSLIPVGPIQWFRGAGPARELIY
NQREGHFPRVTTVSETTRRENMDFSISISNITPADAGTYYCVKFRKGSPD
TEVKSGAGTELSVRAKPSAPVVSGPAARATPGGGGSGGGGSEVQLVESGG
GLVKPGGSLRLSCAASGFTFSNYGISWVRQAPGKGLEWVSSISSGGDNTY
YPDNVKGRFTISRDNAKNSLYLQMNSLRAEDTAVYYCARAGEKAMDYWGQ
GTLVTVSSASTKGPSVFPLAPCSRSTSESTAALGCLVKDYFPEPVTVSWN
SGALTSGVHTFPAVLQSSGLYSLSSVVTVPSSNFGTQTYTCNVDHKPSNT
KVDKTVERKCCVECPPCPAPPVAGPSVFLFPPKPKDTLMISRTPEVTCVV
VDVSHEDPEVQFNWYVDGVEVHNAKTKPREEQFNSTFRVVSVLTVVHQDW
LNGKEYKCKVSNKGLPAPIEKTISKTKGQPREPQVYTLPPSREEMTKNQV
SLTCLVKGFYPSDIAVEWESNGQPENNYKTTPPMLDSDGSFFLYSKLTVD
KSRWQQGNVFSCSVMHEALHNHYTQKSLSLSPGK*
anti-CD40 light chain-linker-SIRPαV2D1
EIVLTQSPATLSLSPGERATLSCRASQTINNNLHWYQQKPGQAPRLLIKY
ASQSISGIPARFSGSGSGTDFTLTISSLEPEDFAVYFCQQFSSWPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGECGGGGSEEELQVIQPDKSVSVAAGESAILHCTVTSLI
PVGPIQWFRGAGPARELIYNQKEGHFPRVTTVSESTKRENMDFSISISNI
TPADAGTYYCVKFRKGSPDTEFKSGAGTELSVRAKPSAPVVSGPAARAT
P*(SEQ ID NO: 18)
Heavy chain of HuCD40-C1H1-V2
EVQLVESGGGLVKPGGSLRLSCAASGFTFSNYGISWVRQAPGKGLEWVSS
ISSGGDNTYYPDNVKGRFTISRDNAKNSLYLQMNSLRAEDTAVYYCARAG
EKAMDYWGQGTLVTVSSASTKGPSVFPLAPCSRSTSESTAALGCLVKDYF
PEPVTVSWNSGALTSGVHTFPAVLQSSGLYSLSSVVTVPSSNFGTQTYTC
NVDHKPSNTKVDKTVERKCCVECPPCPAPPVAGPSVFLFPPKPKDTLMIS
RTPEVTCVVVDVSHEDPEVQFNWYVDGVEVHNAKTKPREEQFNSTFRVVS
VLTVVHQDWLNGKEYKCKVSNKGLPAPIEKTISKTKGQPREPQVYTLPPS
REEMTKNQVSLTCLVKGFYPSDIAVEWESNGQPENNYKTTPPMLDSDGSF
FLYSKLTVDKSRWQQGNVFSCSVMHEALHNHYTQKSLSLSPGK (SEQ
ID NO: 19)
Light chain of HuCD40-C1H1-V2
EIVLTQSPATLSLSPGERATLSCRASQTINNNLHWYQQKPGQAPRLLIKY
ASQSISGIPARFSGSGSGTDFTLTISSLEPEDFAVYFCQQFSSWPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC (SEQ ID NO: 20)
Wild-type SIRPαV2D1-Fc(IgG2)
EEELQVIQPDKSVSVAAGESAILHCTX1TSLIPVGPIQWFRGAGPARELI
YNQX2EGHFPRVTTVSEX3TX4RENMDFSISISNITPADAGTYYCVKFRK
GSPDTEX5KSGAGTELSVRAKPSAPVVSGPAARATPGGGGSGGGGSERKC
CVECPPCPAPPVAGPSVFLFPPKPKDTLMISRTPEVTCVVVDVSHEDPEV
QFNWYVDGVEVHNAKTKPREEQFNSTFRVVSVLTVVHQDWLNGKEYKCKV
SNKGLPAPIEKTISKTKGQPREPQVYTLPPSREEMTKNQVSLTCLVKGFY
PSDIAVEWESNGQPENNYKTTPPMLDSDGSFFLYSKLTVDKSRWQQGNVF
SCSVMHEALHNHYTQKSLSLSPGK* (SEQ ID NO: 21) X1 = V,
X2 = K, X3 = S, X4 = K, X5 = F
EEELQVIQPDKSVSVAAGESAILHCTVTSLIPVGPIQWFRGAGPARELIY
NQKEGHFPRVTTVSESTKRENMDFSISISNITPADAGTYYCVKFRKGSPD
TEFKSGAGTELSVRAKPSAPVVSGPAARATPGGGGSGGGGSERKCCVECP
PCPAPPVAGPSVFLFPPKPKDTLMISRTPEVTCVVVDVSHEDPEVQFNWY
VDGVEVHNAKTKPREEQFNSTFRVVSVLTVVHQDWLNGKEYKCKVSNKGL
PAPIEKTISKTKGQPREPQVYTLPPSREEMTKNQVSLTCLVKGFYPSDIA
VEWESNGQPENNYKTTPPMLDSDGSFFLYSKLTVDKSRWQQGNVFSCSVM
HEALHNHYTQKSLSLSPGK*
SIRPαV2D1M1-Fc(IgG2)
SEQ ID NO: 21, X1 = I, X2 = R, X3 = T, X4 = K,
X5 = F
EEELQVIQPDKSVSVAAGESAILHCTITSLIPVGPIQWFRGAGPARELIY
NQREGHFPRVTTVSETTKRENMDFSISISNITPADAGTYYCVKFRKGSPD
TEFKSGAGTELSVRAKPSAPVVSGPAARATPGGGGSGGGGSERKCCVECP
PCPAPPVAGPSVFLFPPKPKDTLMISRTPEVTCVVVDVSHEDPEVQFNWY
VDGVEVHNAKTKPREEQFNSTFRVVSVLTVVHQDWLNGKEYKCKVSNKGL
PAPIEKTISKTKGQPREPQVYTLPPSREEMTKNQVSLTCLVKGFYPSDIA
VEWESNGQPENNYKTTPPMLDSDGSFFLYSKLTVDKSRWQQGNVFSCSVM
HEALHNHYTQKSLSLSPGK
SIRPαV2D1M2-Fc(IgG2)
SEQ ID NO: 21, X1 = I, X2 = R, X3 = T, X4 = R,
X5 = V
EEELQVIQPDKSVSVAAGESAILHCTITSLIPVGPIQWFRGAGPARELIY
NQREGHFPRVTTVSETTRRENMDFSISISNITPADAGTYYCVKFRKGSPD
TEVKSGAGTELSVRAKPSAPVVSGPAARATPGGGGSGGGGSERKCCVECP
PCPAPPVAGPSVFLFPPKPKDTLMISRTPEVTCVVVDVSHEDPEVQFNWY
VDGVEVHNAKTKPREEQFNSTFRVVSVLTVVHQDWLNGKEYKCKVSNKGL
PAPIEKTISKTKGQPREPQVYTLPPSREEMTKNQVSLTCLVKGFYPSDIA
VEWESNGQPENNYKTTPPMLDSDGSFFLYSKLTVDKSRWQQGNVFSCSVM
HEALHNHYTQKSLSLSPGK
Human CD40-Fc protein
EPPTACREKQYLINSQCCSLCQPGQKLVSDCTEFTETECLPCGESEFLDT
WNRETHCHQHKYCDPNLGLRVQQKGTSETDTICTCEEGWHCTSEACESCV
LHRSCSPGFGVKQIATGVSDTICEPCPVGFFSNVSSAFEKCHPWTSCETK
DLVVQQAGTNKTDVVCGPQDRLREPKSCDKTHTCPPCPAPELLGGPSVFL
FPPKPKDTLMISRTPEVTCVVVDVSHEDPEVKFNWYVDGVEVHNAKTKPR
EEQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNYK
TTPPVLDSDGSFFLYSKLTVDKSRWQQGNVFSCSVMHEALHNHYTQKSLS
LSPGK (SEQ ID NO: 22)
Human CD47-Fc protein
QLLFNKTKSVEFTFCNDTVVIPCFVTNMEAQNTTEVYVKWKFKGRDIYTF
DGALNKSTVPTDFSSAKIEVSQLLKGDASLKMDKSDAVSHTGNYTCEVTE
LTREGETIIELKYRVVSWFSPEPKSCDKTHTCPPCPAPELLGGPSVFLFP
PKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPR
EPQVYTLPPSREEMTKNQVSLTCLVKGFYPSDIAVEWESNGQPENNYKTT
PPVLDSDGSFFLYSKLTVDKSRWQQGNVFSCSVMHEALHNHYTQKSLSLS
PGK (SEQ ID NO: 23)
Human CD172a-Fc protein
MEPAGPAPGRLGPLLCLLLAASCAWSGVAGEEELQVIQPDKSVLVAAGET
ATLRCTATSLIPVGPIQWFRGAGPGRELIYNQKEGHFPRVTTVSDLTKRN
NMDFSIRIGNITPADAGTYYCVKFRKGSPDDVEFKSGAGTELSVRAKPSA
PVVSGPAARATPQHTVSFTCESHGFSPRDITLKWFKNGNELSDFQTNVDP
VGESVSYSIHSTAKVVLTREDVHSQVICEVAHVTLQGDPLRGTANLSETI
RVPPTLEVTQQPVRAENQVNVTCQVRKFYPQRLQLTWLENGNVSRTETAS
TVTENKDGTYNWMSWLLVNVSAHRDDVKLTCQVEHDGQPAVSKSHDLKVS
AHPKEQGSNTAAENTGSNEREPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTP
PVLDSDGSFFLYSKLTVDKSRWQQGNVFSCSVMHEALHNHYTQKSLSLSP
GK (SEQ ID NO: 24)
SIRPα-isoform2-Fc
EEELQVIQPDKSVSVAAGESAILHCTVTSLIPVGPIQWFRGAGPARELIY
NQKEGHFPRVTTVSESTKRENMDFSISISNITPADAGTYYCVKFRKGSPD
TEFKSGAGTELSVRAKPSAPVVSGPAARATPQHTVSFTCESHGFSPRDIT
LKWFKNGNELSDFQTNVDPVGESVSYSIHSTAKVVLTREDVHSQVICEVA
HVTLQGDPLRGTANLSETIRVPPTLEVTQQPVRAENQVNVTCQVRKFYPQ
RLQLTWLENGNVSRTETASTVTENKDGTYNWMSWLLVNVSAHRDDVKLTC
QVEHDGQPAVSKSHDLKVSAHPKEQGSNTAAENTGSNEREPKSCDKTHTC
PPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNK
ALPAPIEKTISKAKGQPREPQVYTLPPSREEMTKNQVSLTCLVKGFYPSD
IAVEWESNGQPENNYKTTPPVLDSDGSFFLYSKLTVDKSRWQQGNVFSCS
VMHEALHNHYTQKSLSLSPGK (SEQ ID NO: 25)
SIRPα-Fc-CD40L
EEELQVIQPDKSVLVAAGETATLRCTATSLIPVGPIQWFRGAGPGRELIY
NQKEGHFPRVTTVSDLTKRNNMDFSIRIGNITPADAGTYYCVKFRKGSPD
DVEFKSGAGTELSVRAKPSAPVVSGPAARATPQHTVSFTCESHGFSPRDI
TLKWFKNGNELSDFQTNVDPVGESVSYSIHSTAKVVLTREDVHSQVICEV
AHVTLQGDPLRGTANLSETIRVPPTLEVTQQPVRAENQVNVTCQVRKFYP
QRLQLTWLENGNVSRTETASTVTENKDGTYNWMSWLLVNVSAHRDDVKLT
CQVEHDGQPAVSKSHDLKVSAHPKEQGSNTAAENTGSNERNIYESKYGPP
CPSCPAPEFLGGPSVFLFPPKPKDTLMISRTPEVTCVVVDVSQEDPEVQF
NWYVDGVEVHNAKTKPREEQFNSTYRVVSVLTVLHQDWLNGKEYKCKVSN
KGLPSSIEKTISKAKGQPREPQVYTLPPSQEEMTKNQVSLTCLVKGFYPS
DIAVEWESNGQPENNYKTTPPVLDSDGSFFLYSRLTVDKSRWQEGNVFSC
SVMHEALHNHYTQKSLSLSLGKHRRLDKIEDERNLHEDFVFMKTIQRCNT
GERSLSLLNCEEIKSQFEGFVKDIMLNKEETKKENSFEMQKGDQNPQIAA
HVISEASSKTTSVLQWAEKGYYTMSNNLVTLENGKQLTVKRQGLYYIYAQ
VTFCSNREASSQAPFIASLCLKSPGRFERILLRAANTHSSAKPCGQQSIH
LGGVFELQPGASVFVNVTDPSQVSHGTGFTSFGLLKL* (SEQ ID NO:
26)
Heavy chain of Selicrelumab
QVQLVQSGAEVKKPGASVKVSCKASGYTFTGYYMHWVRQAPGQGLEWMGW
INPDSGGTNYAQKFQGRVTMTRDTSISTAYMELNRLRSDDTAVYYCARDQ
PLGYCTNGVCSYFDYWGQGTLVTVSSASTKGPSVFPLAPCSRSTSESTAA
LGCLVKDYFPEPVTVSWNSGALTSGVHTFPAVLQSSGLYSLSSVVTVPSS
NFGTQTYTCNVDHKPSNTKVDKTVERKCCVECPPCPAPPVAGPSVFLFPP
KPKDTLMISRTPEVTCVVVDVSHEDPEVQFNWYVDGVEVHNAKTKPREEQ
FNSTFRVVSVLTVVHQDWLNGKEYKCKVSNKGLPAPIEKTISKTKGQPRE
PQVYTLPPSREEMTKNQVSLTCLVKGFYPSDIAVEWESNGQPENNYKTTP
PMLDSDGSFFLYSKLTVDKSRWQQGNVFSCSVMHEALHNHYTQKSLSLSP
GK (SEQ ID NO: 27)
Light chain of Selicrelumab
DIQMTQSPSSVSASVGDRVTITCRASQGIYSWLAWYQQKPGKAPNLLIYT
ASTLQSGVPSRFSGSGSGTDFTLTISSLQPEDFATYYCQQANIFPLTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC (SEQ ID NO: 28)
Cynomolgus CD40-his
EPPTACREKQYLINSQCCSLCQPGQKLVSDCTEFTETECLPCGESEFLDT
WNRETRCHQHKYCDPNLGLRVQQKGTSETDTICTCEEGLHCTSESCESCV
PHRSCLPGFGVKQIATGVSDTICEPCPVGFFSNVSSAFEKCRPWTSCETK
DLVVQQAGTNKTDVVCGPQDRQRHHHHHH (SEQ ID NO: 29)

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A recombinant fusion protein comprising:
(a) an anti-CD40 antibody or an antigen binding portion thereof, comprising a heavy chain variable region, a light chain variable region, a heavy chain constant region and a light chain constant region, and
(b) a CD47 binding domain, wherein the CD47 binding domain is a first Ig-like extracellular domain of human SIRPα isoform 2 (SIRPαV2D1),
wherein the CD47 binding domain is linked to N-terminus of the heavy chain variable region or the light chain variable region, or to C-terminus of the heavy chain constant region or the light chain constant region of the anti-CD40 antibody or the antigen binding portion thereof.

2. The recombinant fusion protein of claim 1, wherein the heavy chain variable region comprises a VH CDR1, a VH CDR2 and a VH CDR3 comprising the amino acid sequences of SEQ ID NOs: 1, 2 and 3, respectively, and the light chain variable region comprises a VL CDR1, a VL CDR2 and a VL CDR3 comprising the amino acid sequences of SEQ ID NOs: 4, 5 and 6, respectively.

3. The recombinant fusion protein of claim 2, wherein the heavy chain variable region comprises an amino acid sequence having at least 80%, 85%, 90%, 91%, 92%, 93%, 94%, 95%, 96%, 97%, 98%, 99% or 100% identity to SEQ ID NO: 7, and the light chain variable region comprises an amino acid sequence having at least 80%, 85%, 90%, 91%, 92%, 93%, 94%, 95%, 96%, 97%, 98%, 99% or 100% identity to SEQ ID NO: 8.

4. The recombinant fusion protein of claim 1, wherein the heavy chain constant region is a human IgG1, IgG2 or IgG4 heavy chain constant region.

5. The recombinant fusion protein of claim 4, wherein the heavy chain constant region comprises the amino acid sequence of SEQ ID NO: 9.

6. (canceled)

7. The recombinant fusion protein of claim 1, wherein the light chain constant region comprises the amino acid sequence of SEQ ID NO: 10.

8. (canceled)

9. (canceled)

10. The recombinant fusion protein of claim 1, wherein the CD47 binding domain is linked to the anti-CD40 antibody or the antigen binding portion thereof via a linker.

11. (canceled)

12. The recombinant fusion protein of claim 10, wherein the linker comprises the amino acid sequence of SEQ ID NOs: 12, 13, 14 or 15.

13. The recombinant fusion protein according to claim 1, wherein the CD47 binding domain is a SIRPαV2D1 variant or a wild-type SIRPαV2D1.

14. The recombinant fusion protein of claim 13, wherein the CD47 binding domain comprises the amino acid sequence of SEQ ID NOs: 11, wherein X1=I, X2=R, X3=T, X4=K, X5=F; X1=I, X2=R, X3=T, X4=R, X5=V; X1=V, X2=K, X3=S, X4=K, X5=F.

15. The recombinant fusion protein according to claim 14, comprising:
a SIRPαV2D1-linker-anti-CD40 heavy chain variable region-heavy chain constant region polypeptide chain comprising the amino acid sequence of SEQ ID NO: 17, wherein X1=I, X2=R, X3=T, X4=K, X5=F; X1=I, X2=R, X3=T, X4=R, X5=V; X1=V, X2=K, X3=S, X4=K, X5=F, and
an anti-CD40 light chain variable region-light chain constant region polypeptide chain comprising the amino acid sequence of SEQ ID NO: 20.

16. The recombinant fusion protein according to claim 15, comprising:
a first polypeptide chain comprising, from N terminus to C terminus, the SIRPαV2D1, the linker, the anti-CD40 heavy chain variable region, and the heavy chain constant region, which comprises the amino acid sequence of SEQ ID NO: 17, wherein X1=I, X2=R, X3=T, X4=K, X5=F; X1=I, X2=R, X3=T, X4=R, X5=V; X1=V, X2=K, X3=S, X4=K, X5=F,
a second polypeptide chain comprising, from N terminus to C terminus, the SIRPαV2D1, the linker, the anti-CD40 heavy chain variable region, and the heavy chain constant region, which comprises the amino acid sequence of SEQ ID NO: 17, wherein X1=I, X2=R, X3=T, X4=K, X5-F; X1=I, X2=R, X3=T, X4=R, X5=V; X1=V, X2=K, X3=S, X4=K, X5=F,
a third polypeptide chain comprising, from N terminus to C terminus, the anti-CD40 light chain variable region and the light chain constant region, which comprises the amino acid sequence of SEQ ID NO: 20, and
a fourth polypeptide chain comprising, from N terminus to C terminus, the anti-CD40 light chain variable region and the light chain constant region, which comprises the amino acid sequence of SEQ ID NO: 20; or
wherein the heavy chain variable region in the first polypeptide chain and the light chain variable region in the third polypeptide chain associate to form an CD40 binding domain,
wherein the heavy chain variable region in the second polypeptide chain and the light chain variable region in the fourth polypeptide chain associate to form an CD40 binding domain,
wherein the heavy chain constant region in the first polypeptide chain and the heavy chain constant region in the second polypeptide chain are associated together.

17. A nucleic acid encoding the recombinant fusion protein according to claim 1.

18. An expression vector comprising the nucleic acid of claim 17.

19. A host cell transformed or transfected with the expression vector of claim 18.

20. A pharmaceutical composition comprising the recombinant fusion protein according to claim 1, and a pharmaceutically acceptable carrier.

21. A method for treating a disease associated with CD40 and/or CD47 signaling in a subject in need thereof, comprising administering to the subject a therapeutically effective amount of the pharmaceutical composition of claim 20.

22. The method of claim 21, wherein the disease is a cancer.

23. The method of claim 22, wherein the cancer is a solid cancer or a hematopoietic cancer.

24. The method of claim 23, wherein the cancer is leiomyosarcoma, acute lymphocytic leukemia (ALL), acute myeloid leukemia (AML), chronic myeloid leukemia (CML), non-Hodgkin's lymphoma, Hodgkin's lymphoma, chronic lymphocytic leukemia, colon cancer, kidney cancer, prostate cancer, cervix cancer, nasopharynx cancer, breast cancer, osteosarcoma, head and neck squamous cell carcinoma, lung cancer (including small cell lung cancer and non-small cell lung cancer), multiple myeloma, melanoma, hepatocellular carcinoma, liver cancer, cholangiocarcinoma, ovarian cancer, bladder cancer, pancreas cancer (including pancreatic ductal adenocarcinoma), or gastric carcinoma.

25. (canceled)


SEQUENCE LISTING (29 sequences)

SEQ ID NO: 1 - protein, 5 aa, synthetic construct
  REGION 1..5: note = VH CDR1 of HuCD40-C1H1-V2
NYGIS

SEQ ID NO: 2 - protein, 17 aa, synthetic construct
  REGION 1..17: note = VH CDR2 of HuCD40-C1H1-V2
SISSGGDNTYYPDNVKG

SEQ ID NO: 3 - protein, 8 aa, synthetic construct
  REGION 1..8: note = VH CDR3 of HuCD40-C1H1-V2
AGEKAMDY

SEQ ID NO: 4 - protein, 11 aa, synthetic construct
  REGION 1..11: note = VL CDR1 of HuCD40-C1H1-V2
RASQTINNNLH

SEQ ID NO: 5 - protein, 7 aa, synthetic construct
  REGION 1..7: note = VL CDR2 of HuCD40-C1H1-V2
YASQSIS

SEQ ID NO: 6 - protein, 9 aa, synthetic construct
  REGION 1..9: note = VL CDR3 of HuCD40-C1H1-V2
QQFSSWPLT

SEQ ID NO: 7 - protein, 117 aa, synthetic construct
  REGION 1..117: note = VH of HuCD40-C1H1-V2
EVQLVESGGGLVKPGGSLRLSCAASGFTFSNYGISWVRQAPGKGLEWVSSISSGGDNTYYPDNVKGRFTISRDNAKNSLYLQMNSLRAEDTAVYYCARAGEKAMDYWGQGTLVTVSS

SEQ ID NO: 8 - protein, 107 aa, synthetic construct
  REGION 1..107: note = VL of HuCD40-C1H1-V2
EIVLTQSPATLSLSPGERATLSCRASQTINNNLHWYQQKPGQAPRLLIKYASQSISGIPARFSGSGSGTDFTLTISSLEPEDFAVYFCQQFSSWPLTFGGGTKVEIK

SEQ ID NO: 9 - protein, 326 aa, synthetic construct
  REGION 1..326: note = Heavy chain constant region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 107 aa, synthetic construct [sequence omitted]

SEQ ID NO: 11 - protein, 131 aa, synthetic construct
  REGION 1..131: note = SIRPalphaV2D1
  VARIANT 27: note = X can be V or I
  VARIANT 53: note = X can be K or R
  VARIANT 66: note = X can be S or T
  VARIANT 68: note = X can be K or R
  VARIANT 103: note = X can be F or V
EEELQVIQPDKSVSVAAGESAILHCTXTSLIPVGPIQWFRGAGPARELIYNQXEGHFPRVTTVSEXTXRENMDFSISISNITPADAGTYYCVKFRKGSPDTEXKSGAGTELSVRAKPSAPVVSGPAARATP

SEQ ID NO: 12 - protein, 5 aa, synthetic construct
  REGION 1..5: note = Linker
GGGGS

SEQ ID NO: 13 - protein, 10 aa, synthetic construct
  REGION 1..10: note = Linker
GGGGSGGGGS

SEQ ID NO: 14 - protein, 15 aa, synthetic construct
  REGION 1..15: note = Linker
GGGGSGGGGSGGGGS

SEQ ID NO: 15 - protein, 20 aa, synthetic construct
  REGION 1..20: note = Linker
GGGGSGGGGSGGGGSGGGGS

SEQ ID NO: 16 - protein, 579 aa, synthetic construct
  REGION 1..579: note = anti-CD40 heavy chain-linker-SIRPalphaV2D1
EVQLVESGGGLVKPGGSLRLSCAASGFTFSNYGISWVRQAPGKGLEWVSSISSGGDNTYYPDNVKGRFTISRDNAKNSLYLQMNSLRAEDTAVYYCARAGEKAMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSAPVVSGPAARATP

SEQ ID NO: 17 - protein, 584 aa, synthetic construct
  REGION 1..584: note = SIRPalphaV2D1-linker-anti-CD40 heavy chain
  VARIANT 27: note = X can be V or I
  VARIANT 53: note = X can be K or R
  VARIANT 66: note = X can be S or T
  VARIANT 68: note = X can be K or R
  VARIANT 103: note = X can be F or V
EEELQVIQPDKSVSVAAGESAILHCTXTSLIPVGPIQWFRGAGPARELIYNQXEGHFPRVTTVSEXTXRENMDFSISISNITPADAGTYYCVKFRKGSPDTEXKSGAGTELSVRAKPSAPVVSGPAARATPGGGGSGGGGSEVQLVESGGGLVKPGGSLRLSCAASGFTFSNYGISWVRQAPGKGLEWVSSISSGGDNTYYPDNVKGRFTISRDNAKNSLYLQMNSLRAEDTAVYYCARAGEKAMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 18 - protein, 350 aa, synthetic construct
  REGION 1..350: note = anti-CD40 light chain-linker-SIRPalphaV2D1
EIVLTQSPATLSLSPGERATLSCRASQTINNNLHWYQQKPGQAPRLLIKYASQSISGIPARFSGSGSGTDFTLTISSLEPEDFAVYFCQQFSSWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSEEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSAPVVSGPAARATP

SEQ ID NO: 19 - protein, 443 aa, synthetic construct
  REGION 1..443: note = Heavy chain of HuCD40-C1H1-V2
EVQLVESGGGLVKPGGSLRLSCAASGFTFSNYGISWVRQAPGKGLEWVSSISSGGDNTYYPDNVKGRFTISRDNAKNSLYLQMNSLRAEDTAVYYCARAGEKAMDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 20 - protein, 214 aa, synthetic construct
  REGION 1..214: note = Light chain of HuCD40-C1H1-V2
EIVLTQSPATLSLSPGERATLSCRASQTINNNLHWYQQKPGQAPRLLIKYASQSISGIPARFSGSGSGTDFTLTISSLEPEDFAVYFCQQFSSWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 369 aa, synthetic construct
  REGION 1..369: note = SIRPalphaV2D1-Fc(IgG2)
  VARIANT 27: note = X can be V or I
  VARIANT 53: note = X can be K or R
  VARIANT 66: note = X can be S or T
  VARIANT 68: note = X can be K or R
  VARIANT 103: note = X can be F or V
EEELQVIQPDKSVSVAAGESAILHCTXTSLIPVGPIQWFRGAGPARELIYNQXEGHFPRVTTVSEXTXRENMDFSISISNITPADAGTYYCVKFRKGSPDTEXKSGAGTELSVRAKPSAPVVSGPAARATPGGGGSGGGGSERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 22 - protein, 405 aa, synthetic construct
  REGION 1..405: note = Human CD40-Fc protein
EPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 353 aa, synthetic construct
  REGION 1..353: note = Human CD47-Fc protein
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 602 aa, synthetic construct
  REGION 1..602: note = Human CD172a-Fc protein
MEPAGPAPGRLGPLLCLLLAASCAWSGVAGEEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNEREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 571 aa, synthetic construct
  REGION 1..571: note = SIRPalpha-isoform2-Fc
EEELQVIQPDKSVSVAAGESAILHCTVTSLIPVGPIQWFRGAGPARELIYNQKEGHFPRVTTVSESTKRENMDFSISISNITPADAGTYYCVKFRKGSPDTEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNEREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 26 - protein, 787 aa, synthetic construct
  REGION 1..787: note = SIRPalpha-Fc-CD40L
EEELQVIQPDKSVLVAAGETATLRCTATSLIPVGPIQWFRGAGPGRELIYNQKEGHFPRVTTVSDLTKRNNMDFSIRIGNITPADAGTYYCVKFRKGSPDDVEFKSGAGTELSVRAKPSAPVVSGPAARATPQHTVSFTCESHGFSPRDITLKWFKNGNELSDFQTNVDPVGESVSYSIHSTAKVVLTREDVHSQVICEVAHVTLQGDPLRGTANLSETIRVPPTLEVTQQPVRAENQVNVTCQVRKFYPQRLQLTWLENGNVSRTETASTVTENKDGTYNWMSWLLVNVSAHRDDVKLTCQVEHDGQPAVSKSHDLKVSAHPKEQGSNTAAENTGSNERNIYESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 27 - protein, 452 aa, synthetic construct
  REGION 1..452: note = Heavy chain of Selicrelumab
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 28 - protein, 214 aa, synthetic construct
  REGION 1..214: note = Light chain of Selicrelumab
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 179 aa, synthetic construct
  REGION 1..179: note = Cynomolgus CD40-his
EPPTACREKQYLINSQCCSLCQPGQKLVSDCTEFTETECLPCGESEFLDTWNRETRCHQHKYCDPNLGLRVQQKGTSETDTICTCEEGLHCTSESCESCVPHRSCLPGFGVKQIATGVSDTICEPCPVGFFSNVSSAFEKCRPWTSCETKDLVVQQAGTNKTDVVCGPQDRQRHHHHHH